(19) United States
(12) Patent Application Publication
(10) Pub. No.: US 2006/0223888 A1
(43) Pub. Date: Oct. 5, 2006

(54) Valproic acid analogues and pharmaceutical composition thereof

(76) Inventors: Frank Slade Abbott, Vancover (CA); Stoyan Kostadinov Karagiozov, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 10/539,049

(22) PCT Filed: Dec. 16, 2003
(86) PCT No.: PCT/CA03/01960

§ 371 Date: Mar. 31, 2006

Related U.S. Application Data
(60) Provisional application No. 60/433,505, filed on Dec. 16, 2002.

(51) Int. Cl. A61K 31/19; C07C 61/06; C07C 61/16; C07C 51/42

(52) U.S. Cl. 514/557; 562/600; 514/573; 562/503; 562/505

(57) ABSTRACT

Analogues of valproic acid useful in treating neuroaffective disorders including convulsions, bipolar disorder, and migraine headache are disclosed. The analogues are halide liver substituted analogues, cyclic analogues, and conjugated diene analogues of valproic acid. Pharmaceutical compositions or prodrugs containing the analogues or pharmaceutically acceptable salts thereof are disclosed. Methods of malting the compounds and treating mammals with neuroaffective disorders are also disclosed.

### RELATED APPLICATIONS

This application claims priority from U.S. Provisional Patent Application Ser. No. 60/433,505 filed 16 Dec. 2002.

### TECHNICAL FIELD

This application relates to analogues of valproic acid, pharmaceutical compositions comprising the analogues, methods of synthesizing the analogues, and uses thereof.

### BACKGROUND

Valproic acid (VPA) and the family of valproate salts are structurally simple drugs that possess a wide range of pharmacological activities. VPA compounds are among the few broad-spectrum anticonvulsants that are effective in both partial and generalized seizures. VPA and the related valproate salts are first line drugs of choice for epilepsy, bipolar disorder, and migraine prophylaxis.

However, despite the excellent efficacy profile of VPA and the related valproate salts, a variety of adverse effects limit their maximum dose and use. While VPA is a relatively safe drug in most patients, it is associated with a rare, frequently fatal, idiosyncratic liver toxicity, with the level of risk greatest in young children under 2 years of age and individuals on polytherapy. In addition, these drugs, like many other anticonvulsants, are also known teratogens and thus their use during pregnancy is limited.

VPA Associated Hepatotoxicity

The primary risk factors of fatal hepatotoxicity associated with VPA therapy are co-administration of other anti-epileptics known to induce cytochrome P450, such as phenyloin or phenobarbital, as well as administration to young age children (less than 2 years old).¹ As a result, reactive metabolites have been implicated in VPA-associated hepatotoxicity.

The VPA derivatives, 4-ene VPA (2-propylpent-4-enoic acid) and (E)-2,4-diene-VPA (2-propylpent-(E)-2,4-dienoic acid) have been demonstrated to induce massive lipid accumulation in rat liver. Expression of 4-ene VPA toxicity has been suggested to require further biotransformation via mitochondrial β-oxidation to (E)-2,4-diene-VPA. The reaction of (E)-2,4-diene VPA, possibly in the CoA thioester form, with glutathione in mitochondria is postulated to produce a localized depletion of glutathione in susceptible individuals that would result in oxidative stress with accompanying hepatocellular damage.

Alternatively, (E)-2,4-diene-VPA may be eventually converted to 3-keto-4-ene VPA, a far more reactive species that inhibits certain β-oxidation enzymes²⁻⁴. (E)-2,4-diene-VPA may also arise from the microsomal cytochrome P450 catalyzed dehydrogenation of the β-oxidation metabolite (E)-2-ene VPA (2-propylpent-2-enoic acid), and the diene metabolite could react with hepatic glutathione through a glucuronide mediated pathway (Scheme 1). 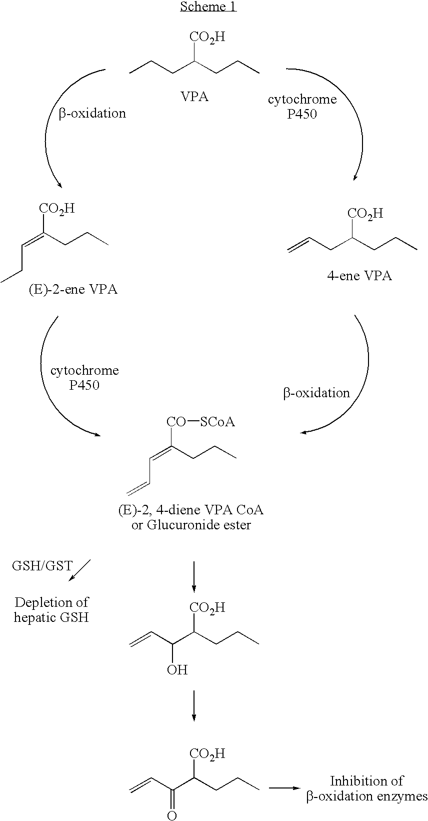
VPA-Induced Teratogenicity

VPA causes neural tube defects leading to spina bifida in 1-2% of children born to mothers treated with VPA.⁵ The risk appears to be related to the dose and peak levels of VPA as well as a history of neural tube defects. Metabolites do not appear to play a role. Structure-teratogenicity studies of VPA analogues indicate that in order to induce teratogenesis, the alpha carbon must be tetrahedral, must be connected to a free carboxylic acid group, and must be connected to one hydrogen atom and to two alkyl groups. Branching of the side chain alkyls reduces teratogenic potency as does the presence of a double bond at the 2-3 position. A double bond at the terminal position of the side chain of VPA maintains teratogenicity. Stereochemistry at the alpha carbon plays a role in the potency of teratogenic analogues.⁶ The mechanism by which VPA produces birth defects is far from clear⁷ and many mechanistic options have been proposed. VPA has been shown to produce reactive oxygen species (ROS) in vivo in the rat⁸ and this can readily be reproduced in rat hepatocytes. The degree of ROS production is highly dependent on the plasma levels of VPA. Induction by phenobarbital and/or chronic administration of VPA to rats leads to significantly increased levels of VPA-induced ROS⁸. The ability of VPA to produce ROS has strong implications towards the mechanism of hepatotoxicity and also for teratogenic properties of this drug. Anticonvulsants, like phenyloin, that are teratogenic are also producers of ROS. The ability of a compound to generate ROS appears to be closely linked to the potential of that compound to cause embryotoxicity⁹. Hence, measurement of the ability of a VPA analogue to produce ROS may be a strong indicator of the teratogenic potential of that compound.

Therefore, there is a need for effective, broad-spectrum anticonvulsants which have reduced or no hepatoxic and teratogenic side effects.

### SUMMARY OF INVENTION

This application discloses analogues of valproic acid and methods of synthesizing and using same. The analogues are useful in the treatment or prophylaxis of conditions responsive to valproic acid therapy, including neuroaffective disorders such as convulsions, epilepsy, bipolar disorder, and migraine headaches.

The analogues comprise compounds represented by the formula (I) and stereoisomers and pharmaceutically acceptable salts thereof. 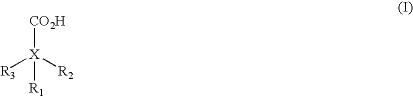
Preferably the analogues comprise between 5 and 13 carbon atoms, wherein X═C and wherein R₁ is optionally present and when present is either H or F.

When R₁ is present, R₂ and R₃ are selected from the group consisting of a linear or branched C1 to C6 alkyl, a linear or branched C2 to C6 n-ene hydrocarbyl (where n=1-5), a linear or branched C1 to C6 n-yne hydrocarbyl (where n=1-5), a linear or branched C1 to C5 ether, a linear or branched C1 to C6 ketone, and —CHₓ-A where A=cyclic C3 to C8 hydrocarbyl and x=0-3. When R₁ is H, at least one of R₂ and R₃ are selectively fluorinated and when R₁ is F, R₂ and R₃ comprise linear or branched alkenyl groups.

In one embodiment when R₁ is not present, R₂ may be H, and there is a double bond between R₃ and X. In this embodiment R₃ is

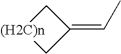

wherein n is 1 to 10.

Alternatively, when R₁ is not present, there is a single bond between X and R₂, and R₂ is 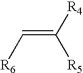
wherein R₄, R₅, and R₆ are selected from the group consisting of H, methyl, ethyl, F, NH₂, cyclopropyl, CF₃, and saturated or unsaturated cyclic (C3 to C8) hydrocarbyl. In this alternative there is a double bond between R₃ and X, and R₃ is 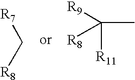
wherein R₇ and R₈ are selected from the group consisting of H, methyl, ethyl, F, NH₂, cyclopropyl and CF₃, and R₉, R₁₀, and R₁₁ are selected from the group consisting of H, methyl, ethyl, F, NH₂, cyclopropyl and CF₃.

Preferably the analogue compounds have between 6 and 10 carbon atoms and may have 8 carbon atoms in one embodiment. The compounds may have multiple sites of alkene or alkyne unsaturation. In one embodiment the compounds may be selectively fluorinated at one or more secondary carbon atoms.

In one embodiment the analogue compounds are dienes having a E, Z configuration. For example, in one embodiment where R₁ is absent and R₂ and R₃ are unsaturated groups, the compounds may have a backbone having the following formula 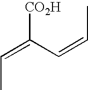
wherein the backbone is optionally substituted by H, F, Me, Et, NH₂, or C1 to C3 hydrocarbyl groups.

This application discloses pharmaceutical compositions containing the valproic acid analogues and their pharmaceutically acceptable salts and prodrugs which are transformable into the valproic acid analogues and salts thereof.

Methods of making the valproic acid analogues and methods of treating neuroaffective disorders using the valproic acid analogues are also disclosed.

### BRIEF DESCRIPTION OF DRAWINGS

FIG. 1 is a concentration time curve of (E,Z)-2,3′-diene VPA levels in rat serum, liver and brain.

### DESCRIPTION

Throughout the following description specific details are set forth in order to provide a more thorough understanding of the invention. However, the invention may be practiced without these particulars. In other instances, well known elements have not been shown or described in detail to avoid unnecessarily obscuring the present invention. Accordingly, the specification and drawings are to be regarded in an illustrative, rather than a restrictive, sense.

This application describes novel analogues of VPA. The compounds are useful in the treatment or prophylaxis of neuroaffective disorders responsive to VPA therapy such as convulsions, epilepsy, bipolar disorder and migraine headaches. The compounds likely exhibit reduced or no hepatotoxic and/or teratogenic side effects compared to VPA. In one embodiment of the invention, the analogues either prevent or reduce the possibility of natural enzymatic processes reducing the carbon backbone at either the 2,3 (or 2′,3′) or 3,4 (or 3′,4′) positions of the compounds. This is achieved through fluorination of the VPA analogues. Blocking the breakage of the carbon backbone results in reduction or elimination of metabolites implicated in the hepatoxicity of VPA. In a second embodiment, the analogues are cyclic VPA analogues. In a third embodiment, the analogues are conjugated analogues with stereodefined double bonds. In a fourth embodiment, the analogues are provided as pharmaceutically acceptable salts or pro-drugs. Methods of making the valproic acid analogues and using them in the treatment or prophylaxis of epilepsy, convulsions, bipolar disorders, migraine headaches, and other neuroaffective disorders are also described herein.

I) Fluorinated VPA Analogues

In a first embodiment of the invention, the compounds are selectively fluorinated analogues of VPA and derivatives thereof. For clarity, the following base, non-fluorinated, structures of VPA analogues to which this embodiment of the invention can be applied are defined by the following base carboxylic acid structure:

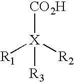

Where:

TABLE 1
VPA Analogue Constituent Structures

| Sub- | | | Further |
|---|---|---|---|
| stituent | Possible | Preferred | Preferred |
| R₁ | H, F, Cl, C1 to C3 | H, F, Me | H |
| | Alkyl, or nothing (in | | |
| | the case of an | | |
| | unsaturated derivative). | | |
| R₂ | Linear or branched C1 | Linear C2 to C4 alkyl; | Propyl, |
| | to C6 alkyl; linear or | linear C2 to C4 n-ene | propenyl, |
| | branched C2 to C6 n- | hydrocarbyl (where | propynyl |
| | ene hydrocarbyl (where | n = 1-5); | |
| | n = 1-5); | linear C1 to C5 ether; | |
| | linear or branched C1 | —CHₓ-A where | |
| | to C5 ether; | A = cyclic | |
| | —CHₓ-A where A = | C3 to C8 | |
| | cyclic C3 to C8 | hydrocarbyl and | |
| | hydrocarbyl and | x = 0-1; | |
| | x = 0-3; | linear C1 to C6 n-yne | |
| | linear or branched C1 | hydrocarbyl (where | |
| | to C6 n-yne | n = 1-5) | |
| | hydrocarbyl | | |
| | (where n = 1-5); | | |
| | Linear or branched C1 | | |
| | to C6 ketones | | |
| R₃ | Linear or branched C1 | Linear C2 to C4 alkyl; | Propyl, |
| | to C6 alkyl; linear or | linear C2 to C4 n-ene | propenyl, |
| | branched C2 to C6 n- | hydrocarbyl (where | propynyl |
| | ene hydrocarbyl (where | n = 1-5); | |
| | n = 1-5); | linear C1 to C5 ether; | |
| | linear or branched C1 | —CHₓ-A where | |
| | to C5 ether; | A = cyclic | |
| | —CHₓ-A where A = | C3 to C8 | |
| | cyclic C3 to C8 | hydrocarbyl and | |
| | hydrocarbyl and | x = 0-1; | |
| | x = 0-3; | linear C1 to C6 n-yne | |
| | linear or branched C1 | hydrocarbyl (where | |
| | to C6 n-yne | n = 1-5) | |
| | hydrocarbyl | | |
| | (where n = 1-5); | | |
| | Linear or branched C1 | | |
| | to C6 ketones | | |

The total number of carbon atoms in the molecule is between 5 and 13. In embodiments of the invention, the total number of carbon atoms in the molecule is between 6 and 10, and in one embodiment the total number of carbon atoms in the molecule is eight.

The compounds of this embodiment of invention are VPA analogues as described in Table 1 above in which one or more of the primary, secondary or tertiary carbon atoms have been halogenated. In a preferred embodiment the halogen used is fluorine and the carbon atom(s) is/are secondary carbon atom(s). The halogenated carbon atom may be of sp² or sp³ hybridization.

Preferably, the halogen in use is fluorine, due to the strength of the carbon-fluorine bond verses the strength of other carbon-halogen bonds. However, the use of other halogens is also contemplated by this invention.

### A) Prevention of 4-ene Metabolite Formation

The compounds of the first embodiment include compounds that are selectively fluorinated at the 4 and/or 4′ position of the carbon backbone so as to prevent the formation of a double bond between the 4 and 5 (or 4′ and 5′) carbon. An example is shown in Scheme 2 where the 4 position is fully fluorinated (4-F₂-VPA). As depicted in Scheme 2, cytochrome P450 (or other enzymes of related function) is unable to cleave the carbon-fluorine bond to form a terminal CH₂═CF— moiety. The formation of a glucuronide analogue along these metabolic pathways is therefore disrupted and the hepatotoxic potential of the VPA analogue is reduced.

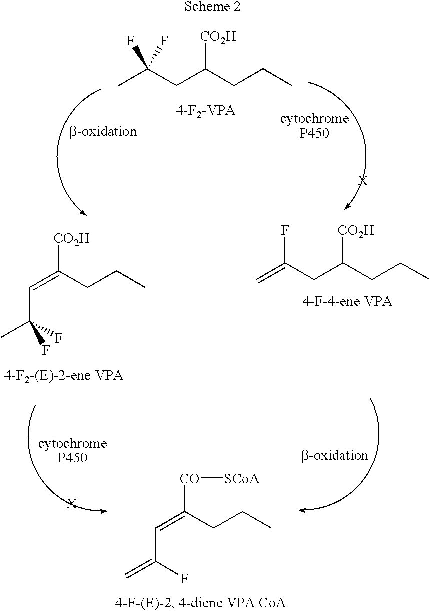

Possible compounds of this embodiment include, but are not limited to:

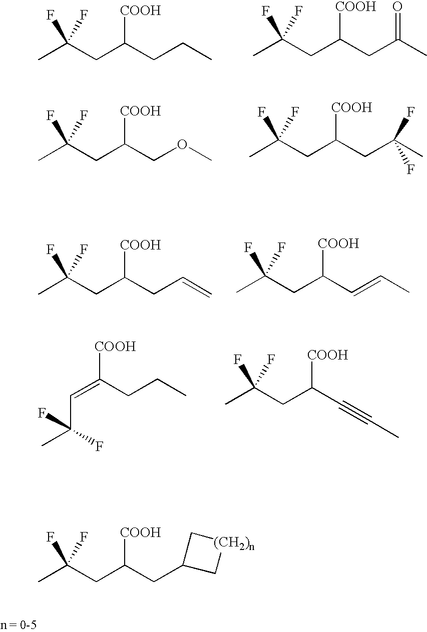

The compounds of the first embodiment also include compounds fluorinated at the terminal (primary) carbon of a propyl/propenyl/propynl carbon chain attached to the carbon at position 2 or 2′. Fluorination in the 5 and/or 5′ position(s) will have a similar effect as fluorination at the 4 and/or 4′-position in preventing 4 (and/or 4′)-ene formation. In preferred embodiments, fluorination at the 5′ position is present in moieties that are at least C4 chains, such that the fluorination occurs at a secondary carbon atom. Compounds contemplated by this embodiment include:

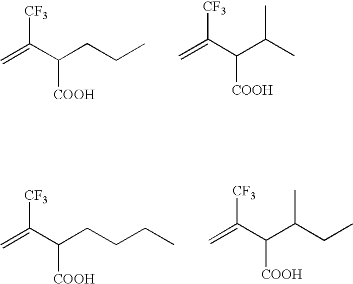

### B) Prevention of 2-ene Metabolite Formation

The compounds of the first embodiment also include VPA analogues that are fluorinated at the 3 and/or 3′ secondary carbon atoms. As depicted in Scheme 3 below, the formation of a glucuronide analogue along these metabolic pathways is disrupted and the hepatotoxic potential of the VPA analogue is therefore reduced.

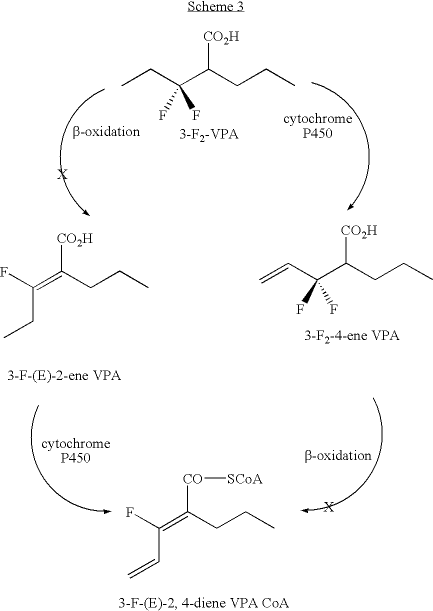

Possible compounds of this embodiment include, but are not limited to:

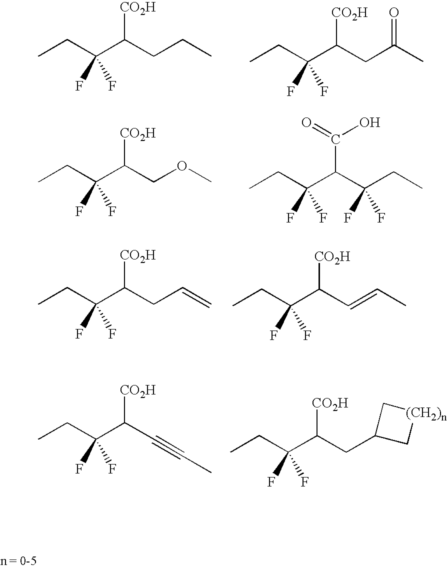

The following analogue and similar compounds that are α-fluorinated are also contemplated by the invention. 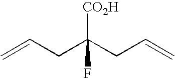
In this embodiment R₂ and R₃ may comprise linear or branched alkenyl groups.

Compounds that are selectively fluorinated at a number of different secondary carbon atom positions are also contemplated within the scope of this invention. Primary and/or tertiary carbon atoms may also be optionally functionalised with fluorine atoms within the same structure. Possible compounds of this embodiment include, but are not limited to:

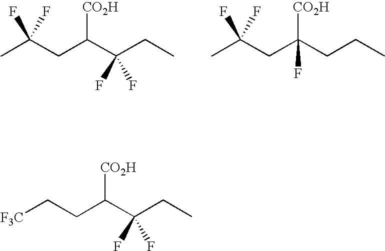

This invention also contemplates compounds with mono-fluorinated secondary carbon atoms. These compounds take advantage of the high stereoselectivity of enzymatic processes. For instance, it is known that cytochrome P450 and other enzymes that carry out oxidation reactions can act with high stereospecificity when cleaving carbon-hydrogen bonds. Compounds that are monofluorinated at secondary carbon atoms may prevent the oxidation of the substrate by enzymes. Some examples of compounds that are contemplated within this embodiment are shown below. However, these compounds should not be considered as limiting the scope of the invention. 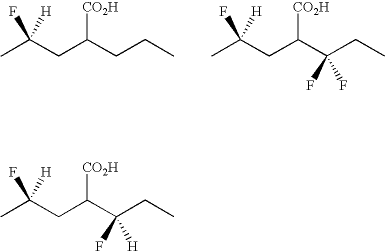
II) Cyclic VPA Analogues

In a second embodiment of the invention, the compounds comprise cyclic VPA analogues. These unsaturated analogues of 2-ene VPA were investigated based on the reported properties of the major metabolite, 2-ene VPA to be less hepatotoxic and embryotoxic than VPA. The results reported by Palaty and Abbott¹³ show that cyclic analogues of 2-ene VPA were more potent than VPA, based on their respective concentrations in brains. Neurotoxicity was less or equivalent to that of VPA. The compounds described by the following structure are also likely useful for treating individuals with epilepsy, or others in need of anticonvulsant therapy.

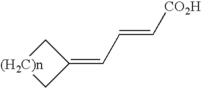

In these compounds n may be between 0 and 10, and is preferably between 4 and 8. In some embodiments, n is 4 or 5. The E isomer is shown above. However both the E and Z isomers are contemplated within the scope of these embodiments. Furthermore, any position on either the dialkenyl chain or the cyclic hydrocarbyl may be optionally functionalised with halogen (particularly F) or C1 to C3 hydrocarbyl group.

III) Conjugated VPA Analogues

In a third embodiment, the invention contemplates VPA analogues containing the (E)-1-(Z)-2′-diene VPA and (E)-1-(E)-2′-diene VPA backbones. These analogues are an extension of the 2-ene VPA analogues and likely have the same beneficial properties, i.e. potency like VPA with reduced liver toxicity and teratogenic properties. A unique finding was that the geometric isomer having the (E)-2-(Z)-3′-diene configuration had greater potency and less neurotoxic effects than the corresponding (E)-2-(E)-3′-diene isomer¹³. The carbon skeletons of these conjugated VPA analogues are shown below:

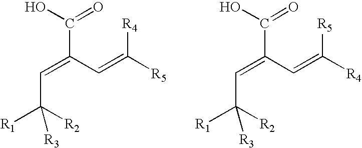

Without limitation, the following R substituents are possible embodiments of such conjugated analogues.

R₁═H, Me, Et, Cyclopropyl, CF₃

R₂, R₃═H, F

R₄═H, Me, Et, F, CF₃, saturated or unsaturated cyclic (C3 to C8) hydrocarbyl

R₅═H

In another embodiment the invention also includes amine substituted conjugated VPA analogues. Some examples of compounds that are contemplated within this embodiment are shown below. However, these compounds should not be considered as limiting the scope of the invention.

| | | |
|---|---|---|
| 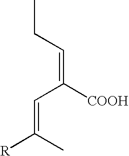 | 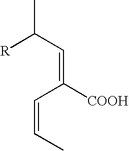 | 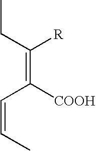 |
| R = F, Me, NH₂ | R = F, Me, NH₂ | R = F, Me, NH₂ |
| 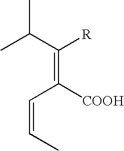 | 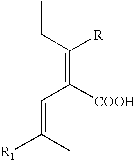 | 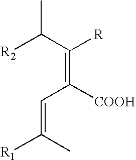 |
| R = F, Me, NH₂ | R = F, Me, NH₂ | R₁, R₂, R₃ = |
| | R₁ = F, Me, NH₂, H | F, Me, NH₂, H |

It will be apparent to those skilled in the art that all of the compounds of the invention described herein may exist in enantiomeric or diastereomeric forms, and that pure enantiomers or diastereomers may be resolved or separated from the racemate or mixture by methods well known in the art. Alternatively, enantiomeric or diastereomeric forms may be prepared by chiral synthesis. R and S enantiomers, racemates, non-racemic mixtures of enantiomers, and mixtures of diastereomers are all contemplated within the scope of this invention.

The VPA analogues described herein may be provided as pharmaceutically acceptable salts or prodrugs. Suitable salts include, but are not limited to, ammonium, sodium, potassium, calcium and magnesium salts. Suitable prodrugs include, but are not limited to, alkyl esters, alkoxy-alkyl esters, hydroxyalkyl esters and amides.

The invention also relates to a method for treating individuals with epilepsy, or for treating others in need of anticonvulsant therapy. Mammals, and in particular humans, who would benefit from this method of treatment include those exhibiting, or at risk of exhibiting, any type of seizure activity. The method of the invention comprises administering to an individual a therapeutically effective amount of at least one compound described herein, or a salt or prodrug thereof, which is sufficient to reduce or prevent seizure activity.

The invention also relates to methods of treating or preventing other neuraffective disorders including bipolar disorder and migraine headaches. The method of the invention comprises administering to an individual a therapeutically effective amount of at least one compound described herein, or a salt or prodrug thereof, which is sufficient to reduce or prevent bipolar disorder, migraine headache, and other neuroaffective disorders.

IV) Examples

The following are examples which are intended to illustrate the embodiments of the invention and which are not intended to limit the scope of the invention.

### EXAMPLE 1

### Chemical Synthesis of Fluorinated VPA Analogues

### A) Preparation of sodium 4,4-difluoro-2-propylpentanoate (7)

The synthetic scheme shown in Scheme 4 below illustrates the synthesis of sodium 4,4-difluoro-2-propylpentanoate 7 and related analogues. The choice of the protecting group²³⁻²⁷ is important to the success of the reaction sequence and the protection of the carbonyl group of 1 under a variety of conditions was investigated. When the acetalization of 1 was carried out using the silylated alcohol Me₃SiO(CH₂)₂OSiMe₃ and a catalytic amount of TMSOTf²⁷ at −78° C. the cyclic acetal 2 was obtained in a quantitative yield. The gem-difluorination of keto ester 4 which is the crucial step of the reaction sequence was carried out under a number of reaction conditions.²⁸⁻³² Fluorination of 4 with DAST^{33,34} or Deoxo-Fluor³⁰⁻³² gave the target ethyl 4,4-difluoro-2-propylpentanoate 5. Scheme 4 below describes ester hydrolysis and preparation of the sodium salts of the end compounds. An alternative approach to the synthesis of 4 is described in Scheme 5.³³⁻³⁵

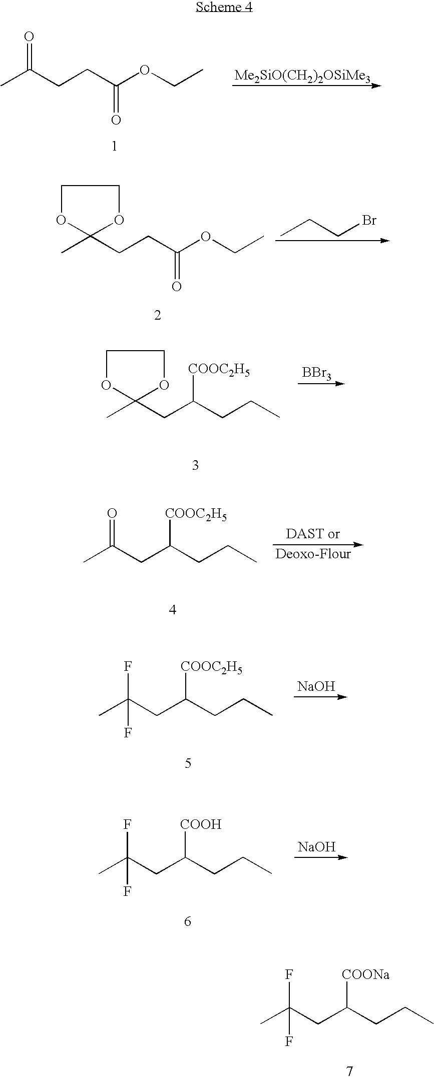

### Preparation of ethyl 3-(2-methyl-1,3-dioxolan-2-yl)propanoate (2)

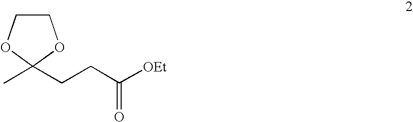

A pre-cooled mixture (−78° C.) of TMSOTf (1.56 g) in CH₂Cl₂ (7 ml) was treated with 1,2-bis(triethylsilyloxy)ethane (16.33 g) and ethyl levulinate (10.09 g) respectively under nitrogen, and the reaction mixture was stirred for 4.5 h. The reaction mixture was treated with pyridine, the organic layer was separated, the aqueous phase was extracted with ethyl acetate, and the combined organic extracts were washed with brine and dried over anhydrous magnesium sulfate and concentrated under reduced pressure. The residue was purified by vacuum distillation to give 12.9 g of compound 2 as a colourless liquid having bp 63° C./0.3 mmHg.

¹H NMR (300 MHz, CDCl₃): 4.03 (q, 2H), 3.84 (m, 4H), 2.29 (t, 2H), 1.92 (t, 2H), 1.23 (s, 3H), 1.16 (t, 3H).

### Preparation of ethyl-2[(2-methyl-1,3-dioxolon-2-yl)methylpentanoate (3)

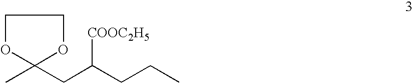

A solution of LDA (7.88 g) in THF (45 ml) was treated with HMPA (12.81 ml) at −78° C. After 30 min a solution of 11.8 g of compound 2 in THF (15 ml) was added for 60 min and stirred for 60 min. Propyl bromide (8.1 ml) in THF (10 ml) was added for 30 min, and the reaction mixture was allowed to warm to room temperature overnight. The reaction mixture was treated with sat. NH₄Cl (150 ml), the organic layer was separated and the water phase was extracted with ethyl acetate. The combined organic extracts were washed with brine and dried over magnesium sulfate. After distilling off the solvent, compound 3 was obtained in 83% yield, bp 82-83° C./2 mmHg.

¹H NMR (300 MHz, CDCl₃): 3.97 (q, 2H), 3.75 (m, 4H), 2.39 (m, 1H), 2.06 (dd, 1H), 1.54 (dd, 1H), 1.44-1.14 (m, 7H), 1.10 (t, 3H), 0.74 (t, 3H).

### Preparation of ethyl 4-oxo-2-propylpentanoate (4)

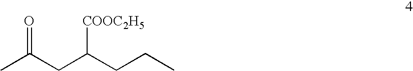

Method 1 (Scheme 4): 9.2 g of compound 3 in hexane (300 ml) were cooled to −60° C. and boron tribromide (1.0 M solution, 60 ml) was added dropwise and the reaction mixture was warmed to −10° C. After stirring for 2 hours it was treated with H₂O (150 ml), and the organic layer was separated. The aqueous layer was extracted with ethyl acetate, and the combined organic layers were washed with brine, dried over magnesium sulfate and concentrated under reduced pressure. The residue was purified by column on silica gel (hexane:ethyl acetate=10:1) to give compound 4 in 87% yield.

Method 2 (Scheme 5): A mixture of cuprous chloride (3.96 g) and palladium(II) chloride (1.4 g) in N,N-dimethylformamide (40 ml) and water (40 ml) was vigorously shaken under oxygen atmosphere until the absorption of oxygen ceased. Compound 22 (6.81 g) (Scheme 5) was added and the reaction mixture was shaken at room temperature for 24 hours. The reaction mixture was poured into 10% HCl (150 ml) and extracted with methylene chloride, dried over magnesium sulfate and concentrated under reduced pressure. After fractionating, a clear colourless liquid of compound 4 was obtained in 65% yield, bp 75-76° C./2 mmHg.

¹H NMR (300 MHz. CDCl₃): 4.01 (q, 2H), 2.75 (m, 2H), 2.38 (m, 1H), 2.04 (s, 3H), 1.51-1.26 (m, 4H), 1.13 (t, 3H), 0.79 (t, 3H).

### Preparation of 4,4-difluoro-2-propylpentanoic acid (6)

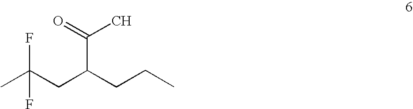

A solution of 2.2 g of compound 4 in methylene chloride (15 ml) was treated with Deoxo-Fluor (3.96 g) and the reaction mixture was stirred at 60° C. for 48 hours. The reaction mixture was cooled to 0° C. and treated with sat. NaHCO₃ until effervescence was completed. The organic layer was separated and the aqueous phase was extracted with CH₂Cl₂. The combined organic layers were dried over magnesium sulfate. The solvent was distilled off and the residue was separated from the unreacted compound 4 by column on silica gel (hexanes:ethyl acetate=20/1). The residue obtained was refluxed with 2.2 N NaOH (25 ml) for 1.5 h. After cooling to 0° C. the reaction mixture was treated with 10% HCl and extracted with ethyl acetate. The organic layer was washed with water and brine, successively, dried over magnesium sulfate and concentrated under reduced pressure. The residue was purified by column on silica gel (hexanes:ethyl=acetate 5:1) to give 460 mg of compound 6.

¹H NMR (300 MHz. CDCl₃): 11.55 (br s, 1H), 2.73-2.63 (m, 1H), 2.39 (m, 1H), 1.99 (m, 1H), 1.94-1.82 (m, 7H), 0.91 (t, 3H).

### Preparation of sodium 4,4-difluoro-2-propylpentanoate (7)

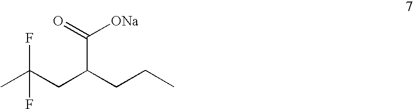

To a solution of 373 mg of compound 6 in methanol (5 ml), 80 mg of sodium hydroxide in methanol (25 ml) was added. The reaction mixture was stirred overnight and the methanol was evaporated under reduced pressure. The residue was washed with ethyl acetate and petroleum ether, successively to give 345 mg of compound 7 as a white powder.

¹H NMR (300 MHz. CDCl₃): 2.33 (m, 2H), 1.81 (t, 1H), 1.57 (t, 3H), 1.55 (m, 1H), 1.47 (m, 3H), 0.91 (t, 3H).

### B) Synthesis of sodium 2-allyl-2-fluoropent-4-enoate (13)

The preparation of target compound 12 and its sodium salt 13 was achieved by fluorination of the precursor 10 with (PhSO₂)₂NF¹⁰ in the presence of LDA in THF at −78° C. to give ester 11 in 39% yield (Scheme 5).

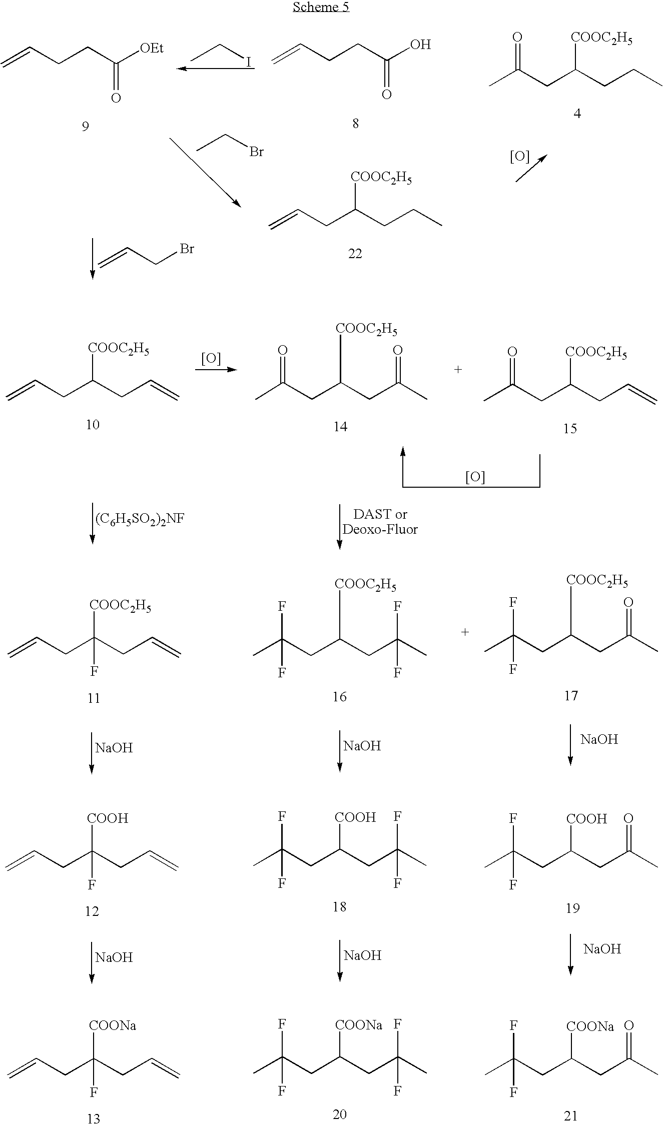

### Preparation of ethyl pent-4-enoate (9)

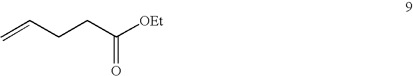

A mixture of 4-pentenoic acid (10.01 g), ethyl iodide (31.19 g), potassium carbonate (10.36 g) and 18-crown-6 (1.28 g) in dry THF (100 ml) was refluxed for 6 hours. After cooling the white solid formed was filtered, and the filtrate was fractionated under reduced pressure to give compound 9 in 80% as a colourless oil, bp 43-44° C./12 mmHg.

### Preparation of ethyl 2-allylpent-4-enoate (10)

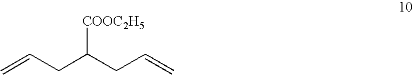

To a pre-cooled at −78° C. solution of LDA (10 g) prepared from diisipropylamine and BuLi (1.6 M in hexane) in tetrahydrofuran (58 ml) was added dropwise HMPA (16.3 ml). After 15 minutes, 10.21 g of compound 9 in THF (10 ml) was slowly added. After 60 minutes, allyl bromide (13.69 g) in THF (10 ml) was added dropwise and the reaction mixture was allowed to warm to room temperature overnight. The reaction mixture was treated with saturated ammonium chloride, extracted with ether, washed with brine, and dried over magnesium sulfate. The mixture was filtered and the solvent distilled off. The residue was purified by vacuum distillation to afford compound 10 in 63% yield as a colourless oil, bp 36-38° C./0.5 mmHg.

### Preparation of ethyl 4-oxo-2-(2-oxopropyl)pentanoate (14) and ethyl 2-(2-oxopropyl)pent-4-enoate (15)

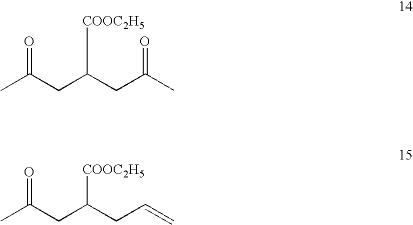

A mixture of cuprous chloride (5.13 g) and palladium(II) chloride (800 mg) in N,N-dimethylformamide (46 ml) and water (3.2 ml) was vigorously shaken under oxygen atmosphere until the absorption of oxygen ceased. 4.41 g of compound 18 was added and the reaction mixture was shaken at room temperature for 24 hours. The reaction mixture was poured into 10% HCl and extracted with methylene chloride, dried over magnesium sulfate and concentrated under reduced pressure. The residue was purified by column on silica gel (hexane:ethyl acetate=10:1) to give compounds 14 and 15 in 24% and 60% yield, respectively as colourless oils.

### Preparation of ethyl 2-(2,2-difluoropropyl)-4,4-difluoropentanoate (16) and ethyl 4,4-difluoro-2-(2-oxopropyl)pentanoate (17)

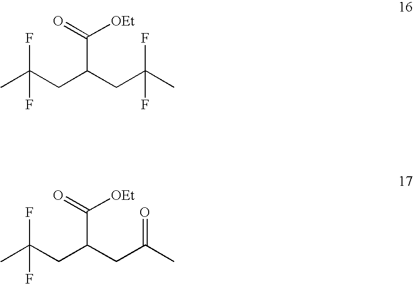

A mixture of 3 g of compound 15 in dry CH₂Cl₂ (15 ml) was treated dropwise with DAST (8 ml) at 0° C. and the reaction mixture was stirred at room temperature for 96 hours. The mixture was then poured into ice-water and the organic layer was separated, the aqueous phase was extracted with CH₂Cl₂, and the combined extracts were dried over magnesium sulfate and concentrated under reduced pressure. The residue was purified by column on silica gel (hexane:ethyl acetate=15:1) to give 367 mg and 864 mg of compounds 16 and 17, respectively as a white solids.

Compound 16:

¹H NMR (300 MHz, CDCl₃): 4.14 (q, 2H), 2.92 (m, 1H), 2.41-2.22 (m, 2H), 2.08-1.89 (m, 2H), 1.59 (t, 6H), 1.22 (t, 3H).

Compound 17:

¹H NMR (300 MHz, CDCl₃): 4.06 (q, 2H), 3.04-2.96 (m, 1H), 2.89-2.81 (m, 1H), 2.69-2.62 (m, 1H), 2.33-2.25 (m, 1H), 2.21 (s, 3H), 2.04-1.88 (m, 1H), 1.54 (t, 3H), 1.16 (t, 3H).

### Preparation of 2-(2,2-difluoropropyl)-4,4-difluoropentanoic acid (18)

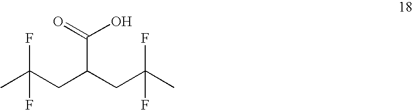

A solution of 157 mg of compound 16 was refluxed with 2.2 N NaOH (25 ml) for 1.5 hours. After cooling to 0° C. the reaction mixture was treated with 10% HCl and extracted with ethyl acetate. The organic layer was washed with water and brine, successively, dried over magnesium sulfate and concentrated under reduced pressure. The residue was purified by column on silica gel (hexanes:ethyl=acetate 5:1) to give 120 mg of compound 18.

¹H NMR (300 MHz. CDCl₃): 11.35 (br s, 1H), 3.04-2.99 (m, 1H), 2.45-2.27 (m, 2H), 2.11-2.05 (m, 2H), 1.62 (t, 6H).

### Preparation of sodium 2-(2,2-difluoropropyl)-4,4-difluoropentanoate (20)

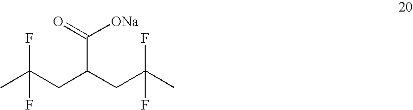

To a solution of 329 mg of compound 18 in methanol (5 ml), 58 mg of sodium hydroxide in methanol (20 ml) was added. The reaction mixture was stirred overnight and the methanol was evaporated under reduced pressure. The residue was washed with ethyl acetate and petroleum ether, successively, to give 323 mg of compound 20 as a white powder.

### Preparation of 4,4-difluoro-2-(2-oxopropyl)pentanoic acid (19)

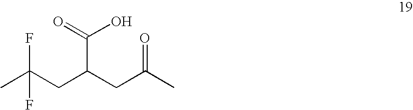

A solution of 805 mg of compound 17 was refluxed with 2.2 N NaOH (25 ml) for 1.5 hours. After cooling to 0° C. the reaction mixture was treated with 10% HCl and extracted with ethyl acetate. The organic layer was washed with water and brine, successively, dried over magnesium sulfate and concentrated under reduced pressure. The residue was purified by column on silica gel (hexanes:ethyl=acetate 3:1) to give 371 mg of compound 19.

### Preparation of sodium 4,4-difluoro-2-(2-oxopropyl)pentanoate (21)

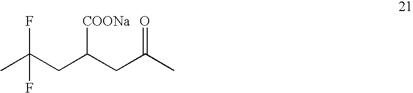

To a solution of 303 mg of compound 19 in methanol (5 ml), 60 mg of sodium hydroxide in methanol (30 ml) was added. The reaction mixture was stirred overnight and the methanol was evaporated under reduced pressure. The residue was washed with ethyl acetate and petroleum ether, successively, to give 300 mg of compound 21 as a white powder.

### Preparation of ethyl 2-allyl-2-fluoropent-4-enoate (11)

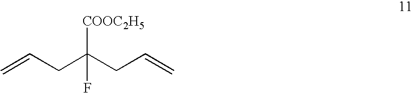

To a pre-cooled (at −78° C.) solution of LDA (2.37 g) in tetrahydrofuran (30 ml) was added dropwise HMPA (3.85 ml). After stirring for 15 min a solution of 2.6 g of compound 10 in tetrahydrofuran (10 ml) was added over 1.5 h and stirred for 30 minutes. A solution of N-fluorobenzene sulfonimide (8.2 g) in tetrahydrofuran (30 ml) was then added dropwise and the reaction mixture was allowed to warm to room temperature overnight. The reaction mixture was treated with saturated ammonium chloride and 10% HCl, respectively. The organic layer was separated and the aqueous phase was extracted with ether. The combined organic extracts were washed with brine, and dried over magnesium sulfate and concentrated under reduced pressure. The residue was purified by column on silica gel (hexane:ethyl acetate=30:1 to 20:1) to give 1.1 g of compound 11.

### Preparation of 2-Allyl-2-fluoropent-4-enoic acid (12)

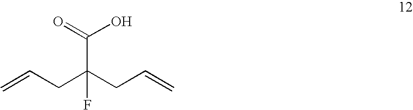

A mixture of 1.1 g of compound 11 and 2.2 N sodium hydroxide (35 ml) was heated at 60° C. for 96 hours. The reaction mixture was cooled to 0° C. and treated with 10% HCl until pH 1 was attained. The mixture was then extracted with ethyl acetate, dried over magnesium sulfate and concentrated under reduced pressure. The residue was purified by column on silica gel (hexane:ethyl acetate=10:1) to give 592 mg of compound 12.

¹H NMR (300 MHz. CD₃OD): 5.79 (m, 2H), 5.16 (d, 4H), 2.64 (d, 2H), 2.55 (d, 2H).

### Preparation of sodium 2-allyl-2-fluoropent-4-enoate (13)

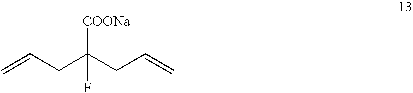

A mixture of 818 mg of compound 12 in methanol and 195 mg of sodium hydroxide in methanol (55 ml) was stirred overnight and the methanol was evaporated under reduced pressure. The residue was washed with ethyl acetate and petroleum ether, successively, to give 859 mg of compound 13 as a white solid.

¹H NMR (300 MHz. CD₃OD): 5.84 (m, 2H), 5.09 (td, 4H), 2.71-2.41 (m, 4H).

### C) Synthesis of sodium (2Z)-4,4-difluoro-2-propylpent-2-enoate (29)

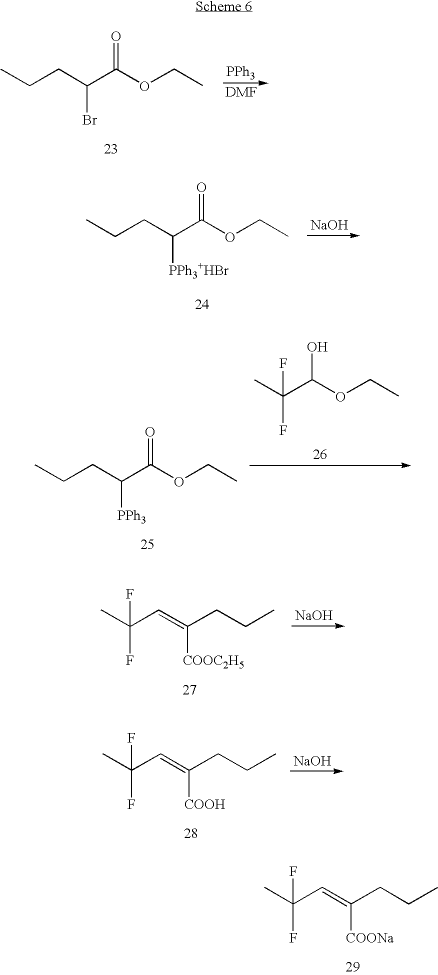

### Preparation of ethyl 2-(triphenylphosphoranylidene)pentanoate (25)

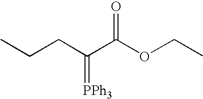

Compound 25 was prepared according to the procedure described in U.S. Pat. No. 4,965,401 (1990).

### Preparation of ethyl (2E)-4,4-difluoro-2-propylpent-2-enoate (27)

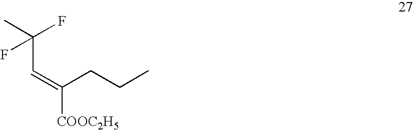

A mixture of 510 mg of compound 25, pTSA (19 mg) and ethyl 2,2-difluoropropanoate 25⁴⁸ in methylene chloride (12 ml) was refluxed for 24 hours. The solid formed was filtered and concentrated under reduced pressure. The residue was purified by column on silica gel (hexane:ethyl acetate=15:1) to give 810 g of compound 27.

¹H NMR (300 MHz. CDCl₃): 6.60 (t, 1H), 4.21 (q, 2H), 2.41 (t, 2H), 1.74 (t, 3H), 1.45 (dq, 2H), 1.29 (t, 3H), 0.92 (t, 3H).

### Preparation of (2Z)-4,4-difluoro-2-propylpent-2-enoic acid (28)

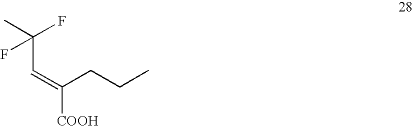

A mixture of 252 mg of compound 27 and 2.2 N sodium hydroxide (3 ml) was heated at 60° C. for 2 hours. The reaction mixture was concentrated under reduced pressure and treated with 1N HCl. The mixture was extracted with ethyl acetate, washed with brine, dried over magnesium sulfate and concentrated under reduced pressure. The residue was purified by column on silica gel (hexane:ethyl acetate=10:1) to give 198 mg of compound 28.

¹H NMR (300 MHz, CDCl₃): 12.17 (br s, 1H), 6.76 (t, 1H), 2.42 (t, 2H), 1.75 (t, 3H), 1.49 (dq, 2H), 0.94 (t, 3H).

### Preparation of sodium (2Z)-4,4-difluoro-2-propylpent-2-enoate (29)

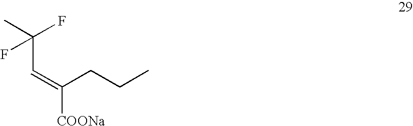

Compound 29 was prepared according to the procedure described above for the preparation of sodium 4,4-difluoro-2-propylpentanoate (compound 7).

### D) Synthesis of sodium 2-propyl-3-(trifluoromethyl)but-3-enoate (32)

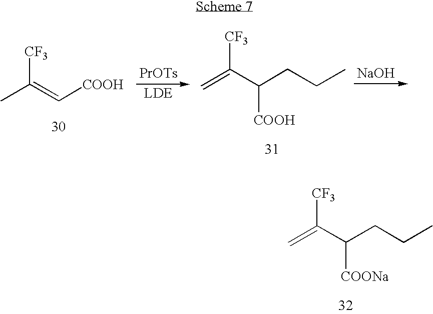

The preparation of 2-propyl-3-(trifluoromethyl)but-3-enoic acid 31 and its sodium salt 32 outlined in Scheme 7 above illustrates the approach to the synthesis of VPA analogues containing a CF₃-group.³⁶⁻³⁸

### EXAMPLE 2

### Synthesis of Cyclic VPA Analogues

The Wittig reaction and its modification, the base-promoted Horner-Wadsworth-Emmons olefination of aldehydes and ketones with phosphonate carbanions, is a widely employed approach to the synthesis of α,β-unsaturated ester⁴¹. In order to synthesize selected substituted (2E)- and (2Z)-4-substituted but-2-enoic acids, the inventors' strategy was based on the coupling of the β,β-disubstituted α,β-unsaturated aldehydes 38 and the generated phosphonate carbanions under specific conditions that provide high E- and Z-stereoselectivity (Scheme 8).

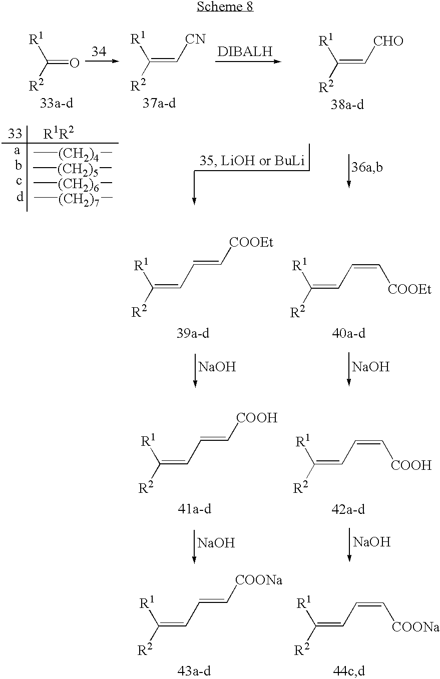

The starting compounds 38 were obtained by olefination of ketones 33 with diethyl cyanomethyl phosphonate 34 carried out in ether or DMF to provide nitriles 37^{42a}. Further reduction with DIBALH carried out in pentane or ether, gave known aldehydes 38a-c^{42b} (Scheme 8). Reduction of nitrile 37d, performed in pentane or ether afforded aldehyde 38d.

The stereoselective conversion of aldehydes 38 to (E)-α,β-unsaturated esters 39 was performed with triethyl phosphonoacetate 35⁴³⁻⁴⁴ in the presence of LiOH or BuLi. The stereoselective synthesis of (2Z)-4-cycloalkylidenebut-2-enoates 40 was achieved by Horner-Wadsworth-Emmons olefination performed on aldehydes 38 with ethyl (diphenylphosphono)acetate 36a and ethyl (di-o-tolylphosphono)acetate 36b, respectively, in the presence of Triton B in tetrahydrofuran.

Further basic hydrolysis of 40 under mild conditions afforded the corresponding (2Z)-4-cycloalkylidenebut-2-enoic acids 42 in excellent yields. The (Z)-acids 42 were converted to the corresponding sodium salts 44 by treatment with NaOH in MeOH.

### General Procedure for the Preparation of Ethyl (2E)-4-Cycloalkylidenebut-2-enoates (39a-d)

Method A: A suspension of LiOH.H₂O (2.2 mmol) in anhyd THF (4 mL) was treated at room temperature with 35 (2.2 mmol) followed by aldehyde 38 (2 mmol) and stirred over 2.5 or 16 hours. The mixture was filtered through silica gel and washed with ether. The filtrate was concentrated under reduced pressure and the residue was purified by column (hexanes/ether=100:1.5) to afford esters 39d as a colourless liquid.

Method B: To a solution of triethyl phosphonoacetate 35 (30.06 mmol) in THF under argon at 0° C. was added DMPU (7.58 ml) over 10 min and BuLi (21.8 ml) over 20 min and the stirring was continued for 20 minutes. The solution was then cooled to −78° C. and a solution of aldehyde 38 (17.0 mmol) in THF was added dropwise over 1.5 h, stirred for 1 h and the reaction mixture was allowed to warm to 0° C. over 1.5 h. The reaction was quenched with sat. NH₄Cl solution, extracted with ethyl acetate, washed successively with H₂O and brine, dried (MgSO₄) and concentrated under reduced pressure. The residue was purified by column chromatography on silica gel to yield compounds 39.

### Ethyl (2E)-4-Cyclopentylidenebut-2-enoate (39a)

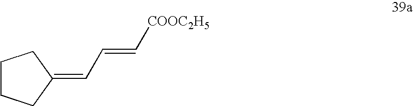

Procedure A: Yield: 65%),

Procedure B: Yield: 50%.

¹H NMR (300 MHz, CDCl₃): 7.35 (dd, 1H), 6.0 (d, 1H), 5.62 (d, 1H), 4.11 (q, 2H), 2.41 (t, 2H), 2.31 (t, 2H), 1.68-1.57 (m, 4H), 1.24 (t, 3H);

### Ethyl (2E)-4-Cyclohexylidenebut-2-enoate (39b)

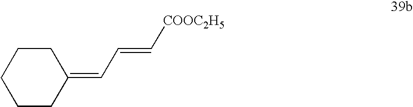

Procedure A: Yield: 66%.

Procedure B: Yield: 71%.

¹H NMR (300 MHz, CDCl₃): 7.55 (dd, 1H), 5.89 (d, 1H), 5.74 (d, 1H), 4.15 (q, 2H), 2.41-2.29 (m, 2H), 2.21-2.11 (m, 2H), 1.58-1.54 (m, 6H), 1.24 (t, 3H).

### Ethyl (2E)-4-Cycloheptylidenebut-2-enoate (39c)

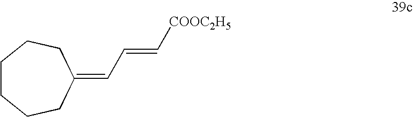

Procedure A: Yield: 41%.

Procedure B: Yield: 64%.

¹H NMR (300 MHz, CDCl₃): 7.56 (dd, 1H), 5.96 (d, 1H), 5.79 (d, 1H), 4.18 (q, 2H), 2.51 (t, 2H), 2.33 (t, 2H), 1.69-1.61 (m, 3H), 1.50-1.41 (m, 5H), 1.27 (t, 3H).

### Ethyl (2E)-4-Cyclooctylidenebut-2-enoate (39d)

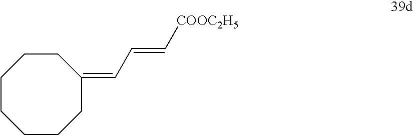

Procedure A: Yield: 58%.

Procedure B: Yield 70%)

¹H NMR (300 MHz, CDCl₃): 7.56 (dd, 1H), 5.96 (d, 1H), 5.69 (d, 1H), 4.16 (q, 2H), 2.41 (t, 2H), 2.25 (t, 2H), 1.69-1.64 (m, 4H), 1.48-1.39 (m, 6H), 1.25 (t, 3H);

### General Procedure for the Preparation of Ethyl (2Z)-4-Cycloalkylidenebut-2-enoates (40a-d)

To a solution of 36a/36b (1 mmol) in THF (3 mL) at −78° C. under argon was added dropwise Triton B (benzyltriethylammonium hydroxide 40% in MeOH) (0.54 mL, 1.35 mmol) over 15 minutes. After 30 minutes, a solution of aldehyde 38 (1.1 mmol) in THY (1 mL) was added dropwise for 20 minutes and the resulting mixture was gradually warmed to 0° C. The reaction was quenched with sat. NH₄Cl solution extracted with ethyl acetate and the combined organic layers were washed successively with H₂O and brine, dried (MgSO₄) and then concentrated in vacuo. The crude residue was purified on chromatotron (silica gel, hexanes followed by hex-anes/Et₂O 100:1.5) to yield a mixture of (Z/E) products 40/39 determined by ¹H HMR analysis. Further separation of the mixture afforded analytical samples of compounds 40 and 39, respectively, as colourless liquids.

### Ethyl (2Z)-4-Cyclopentylidenebut-2-enoate (40a)

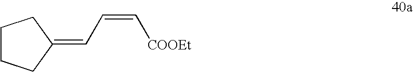

Yield: From 36a and 36b: 47% and 56% respectively.

¹H NMR (300 MHz, CDCl₃): 7.21 (d, 1H), 6.65 (t, 1H), 5.44 (d, 1H), 4.09 (q, 2H), 2.36 (t, 4H), 1.67-1.56 (m, 4H), 1.2 (t, 3H).

### Ethyl (2Z)-4-Cyclohexylidenebut-2-enoate (40b)

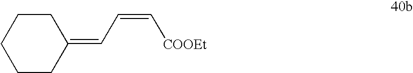

Yield: From 36a and 36b: 61% and 67%, respectively.

¹H NMR (300 MHz, CDCl₃): 7.08 (d, 1H), 6.84 (t, 1H), 5.47 (d, 1H), 4.09 (q, 2H), 2.27 (br t, 2H), 2.18 (br t, 2H), 1.51-1.49 (m, 6H), 1.19 (t, 3H);

### Ethyl (2Z)-4-Cycloheptylidenebut-2-enoate (40c)

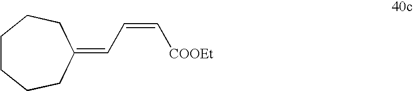

Yield: From 36a and 36b: 45% and 76%, respectively.

¹H NMR (300 MHz, CDCl₃): 7.16 (d, 1H), 6.87 (t, 1H), 5.53 (d, 1H), 4.16 (q, 2H), 2.47 (t, 2H), 2.39 (t, 2H), 1.62-1.55 (m, 4H), 1.51-1.46 (m, 4H), 1.27 (t, 3H);

### Ethyl (2Z)-4-Cyclooctylidenebut-2-enoate (40d)

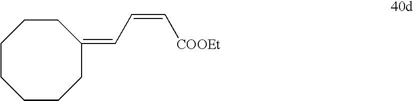

Yield: From 36a and 36b: 48% and 82%, respectively.

¹H NMR (300 MHz, CDCl₃): 7.21 (d, 1H), 6.87 (t, 1H), 5.51 (d, 1H), 4.14 (q, 2H), 2.38 (t, 2H), 2.32 (t, 2H), 1.70-1.66 (m, 4H), 1.49-1.44 (m, 6H), 1.25 (t, 3H);

### General Procedure for Preparation of (2E)-4-Cycloalkylidenebut-2-enoic Acids (41a-d) and (2Z)-4-Cycloalkylidenebut-2-enoic Acids (42a-d)

A mixture of esters (E)-39 or (Z)-40 (0.44 mmol) and NaOH (1.2 g, 30 mmol) in H₂O/MeOH (7.8/3.9 mL) was gently refluxed for 45 min. After cooling the reaction mixture was diluted with brine (5 mL) and extracted with ether. The aqueous layer was acidified with 10% HCl, extracted with ethyl acetate), and the combined extracts washed with brine, dried over MgSO₄ and concentrated in vacuo. Chromatotron chromatography (silica gel, hexanes/EtOAc 90:10) afforded pure acids (E)-41 or (Z)-42 respectively as a white solid.

### (2E)-4-Cyclopentylidenebut-2-enoic Acid (41a)

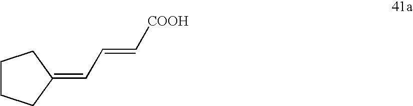

Yield: 60%, mp 112-114° C.

¹H NMR (300 MHz, CDCl₃): 7.43 (dd, 1H), 6.12 (d, 1H), 5.67 (d, 1H), 2.48 (t, 2H), 2.39 (t, 2H), 1.79-1.63 (m, 4H).

IR (KBr): v=1679 cm⁻¹.

### (2E)-4-Cyclohexylidenebut-2-enoic Acid (41b)

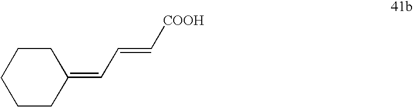

Yield: 80%, mp 132-134° C.

¹H NMR (300 MHz, CDCl₃): 7.64 (dd, 1H), 5.99 (d, 1H), 5.75 (d, 1H), 2.39 (br s, 2H), 2.26 (br s, 2H), 1.61 (br d, 6H);

IR (KBr): v=1680 cm⁻¹.

### (2E)-4-Cycloheptylidenebut-2-enoic Acid (41c)

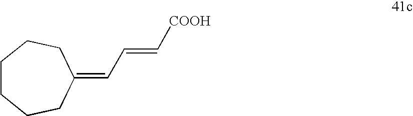

Yield: 85%, mp 88-89° C.

¹H NMR (300 MHz, CDCl₃): 7.58 (dd, 1H), 6.02 (d, 1H), 5.74 (d, 1H), 2.53 (t, 2H), 2.32 (t, 2H), 1.75 (br d, 4H), 1.51 (br d, 4H).

IR (KBr): v=1680 cm⁻¹.

### (2E)-4-Cyclooctylidenebut-2-enoic Acid (41d)

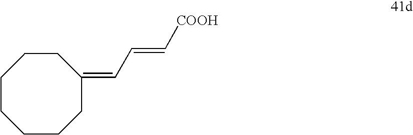

Yield: 89%, mp 114-116° C.

¹H NMR (300 MHz, CDCl₃): 7.62 (dd, 1H), 6.07 (d, 1H), 5.72 (d, 1H), 2.47 (t, 2H), 2.32 (t, 2H), 1.75 (br d, 4H), 1.51 (br d, 6H).

IR (KBr): v=1680 cm⁻¹.

### (2Z)-4-Cyclopentylidenebut-2-enoic Acid (42a)

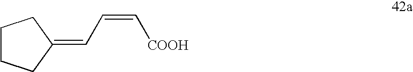

Yield: 63%, mp 105-107° C.

¹H NMR (300 MHz, CDCl₃): 7.19 (d, 1H), 6.79 (t, 1H), 5.49 (d, 1H), 2.47-2.39 (m, 4H), 1.79-1.62 (m, 4H).

IR (KBr): v=1684 cm⁻¹.

### (2Z)-4-Cyclohexylidenebut-2-enoic Acid (42b)

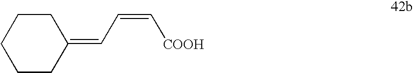

Yield: 68%, mp 121-123° C.

¹H NMR (300 MHz, CDCl₃): 7.06 (d, 1H), 7.0 (d, 1H), 5.53 (d, 1H), 2.39 (br t, 2H), 2.34 (br t, 2H), 1.61 (br s, 6H).

IR (KBr): v=1680 cm⁻¹.

### (2Z)-4-Cycloheptylidenebut-2-enoic Acid (42c)

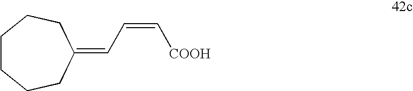

Yield: 86%, mp 86-88° C.

¹H NMR (300 MHz, CDCl₃): δ=7.12 (d, 1H), 6.94 (t, 1H), 5.53 (d, 1H), 2.51 (t, 2H), 2.39 (t, 2H), 1.65-1.64 (m, 4H), 1.54-1.52 (m, 4H).

IR (KBr): v=1684 cm⁻¹.

### (2Z)-4-Cyclooctylidenebut-2-enoic Acid (42d)

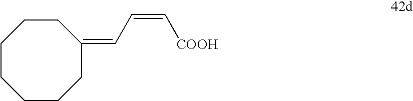

Yield: 64%, mp 110-112° C.

¹H NMR (300 MHz, CDCl₃): 7.16 (d, 1H), 6.97 (t, 1H), 5.53 (d, 1H), 2.45 (t, 2H), 2.33 (t, 2H), 1.79-1.68 (m, 4H, 1.58-1.46 (m, 6H).

IR (KBr): v=1684 cm⁻¹.

### General Procedure for the Preparation of Sodium (2E)-4-Cycloalkylidenebut-2-enoates (43a-d) and Sodium (2Z)-4-Cycloalkylidenebut-2-enoates (44c,d)

To a solution of acid (E)-41 or (Z)-42 (2.4 mmol) in MeOH (10 mL) was added dropwise a solution of NaOH (2.18 mmol) in MeOH (20 mL) at 0° C. under argon and the resulting mixture was warmed to room temperature overnight. The MeOH was concentrated under reduced pressure and the white solid formed was filtered, washed successfully with ether, and dried in vacuo to give pure sodium salt ((E)-43)/((Z)-44) as a white solid; mp>300° C.

### Sodium (2E)-4-Cyclopentylidenebut-2-enoate (43a)

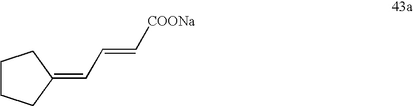

Yield: 87%, ¹H NMR (400 MHz, CD₃OD): 7.35 (dd, 1H), 6.03 (d, 1H), 5.73 (d, 1H), 2.46 (t, 2H), 2.36 (t, 2H), 1.73-1.63 (m, 4H).

### Sodium (2E)-4-Cyclohexylidenebut-2-enoate (43b)

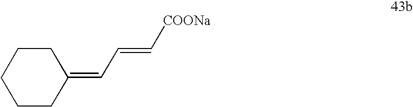

Yield: 90%, ¹H NMR (400 MHz, CD₃OD): 7.40 (dd, 1H), 6.0 (d, 1H), 5.8 (d, 1H), 2.4 (br s, 2H), 2.2 (br s, 2H), 1.6 (br s, 6H).

### Sodium (2E)-4-Cycloheptylidenebut-2-enoate (43c)

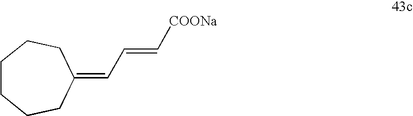

Yield: 77%, ¹H NMR (400 MHz, CD₃OD): 7.35 (dd, 1H), 5.93 (d, 1H), 5.78 (d, 1H), 2.51 (t, 2H), 2.33 (t, 2H), 1.73-1.63 (m, 4H), 1.57-1.52 (m, 4H).

### Sodium (2E)-4-Cyclooctylidenebut-2-enoate (43d)

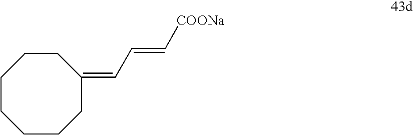

Yield: 76%, ¹H NMR (400 MHz, CD₃OD): 7.39 (dd, 1H), 5.97 (d, 1H), 5.77 (d, 1H), 2.45 (t, 2H), 2.28 (t, 2H), 1.78-1.71 (m, 4H), 1.66-1.51 (m, 6H).

### Sodium (2Z)-4-Cycloheptylidenebut-2-enoate (44c)

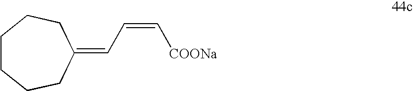

Yield: 94%, ¹H NMR (300 MHz, CD₃OD): 6.97 (d, 1H), 6.51 (d, 1H), 5.65 (d, 1H), 2.44 (t, 2H), 2.34 (t, 2H), 1.63-1.59 (m, 4H), 1.56-1.51 (m, 4H).

### Sodium (2Z)-4-Cyclooctylidenebut-2-enoate (44d)

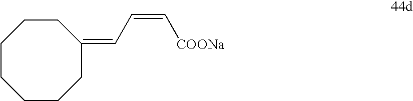

Yield: 92%, ¹H NMR (300 MHz, CDCl₃): 7.04 (d, 1H), 6.56 (d, 1H), 5.48 (d, 1H), 2.39 (t, 2H), 2.29 (t, 2H), 1.71 (m, 4H), 1.51 (br s, 6H).

### EXAMPLE 3

### Synthesis of Conjugated VPA Analogues

The synthesis of the target compound 50 is outlined in Scheme 9. The starting ester 46 was prepared from trans-2-pentenoic acid 45 by refluxing with an excess of ethyl alcohol in the presence of catalytic amounts of conc. H₂SO₄ in benzene. The ester 46 was allowed to react with isobutyraldehyde in the presence of LDA in tetrahydrofuran to afford alcohol 47. Further mesylation of 47 with MsCl followed by elimination under basic conditions gave ester 48. Upon basic hydrolysis of 48, the acid 49 obtained was converted into its sodium salt 50 under the conditions described above for the preparation of the other sodium salts.

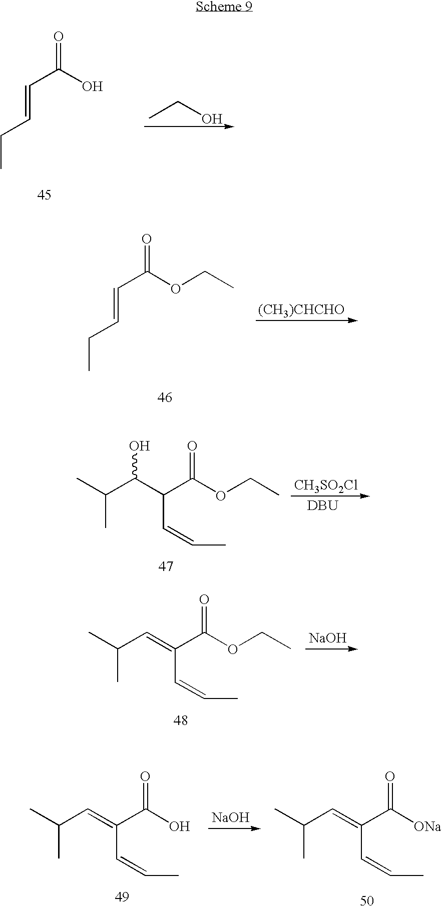

### Preparation of ethyl (3Z)-2-(1-hydroxy-2-methylpropyl)pent-3-enoate (47)

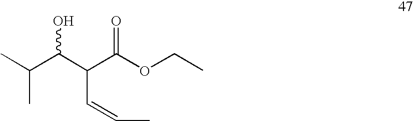

8.18 g (bp 80-81° C./8 mmHg) of compound 47 were prepared according to the procedure described for compound 10 from 6.41 g of ethyl (2Z)-pent-2-enoate and a solution of isobutyraldehyde (3.61 g) in tetrahydrofuran (7 ml) and a standard work-up procedure under acidic conditions.

### Preparation of (2E)-4-methyl-2-[(1Z)-prop-1-enyl])pent-2-enoic acid (49)

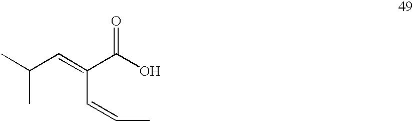

Step A: A mixture of 7.98 g of compound 47 and triethylamine (8.77 ml) in methylene chloride (66 ml) was treated dropwise with a solution of methanesulfonyl chloride (3.3 ml) in methylene chloride (4 ml) at 0° C. After 60 minutes the reaction mixture was filtered and concentrated under reduced pressure. The residue was dissolved in tetrahydrofuran (55 ml) and treated with a solution of DBU (6 ml). After refluxing for 2 hours the mixture was cooled, treated with H₂O (35 ml), extracted with ether, washed with brine, and dried over magnesium sulfate.

Step B: The ether 48 was evaporated under reduced pressure, and the crude residue was treated with 3N NaOH/H₂O (18.0 ml:9.0 ml) and heated at 60° C. for 48 hours. The mixture was then cooled, extracted with ether, and the aqueous phase was acidified with 10% HCl, extracted with ethyl acetate, dried over magnesium sulfate, and evaporated under reduced pressure. The residue was purified by column on silica gel (hexane:ether=9:1) to give 4.01 g of compound 49.

¹H NMR (300 MHz, CDCl₃): 11.95 (s, 1H), 6.73 (d, 1H), 5.91 (d, 1H), 5.77 (m, 1H), 2.54 (m, 1H), 1.55 (d, 3H), 0.97 (d, 6H).

### Preparation of sodium (2E)-4-methyl-2-[(1Z)-prop-1-enyl])pent-2-enoate (50)

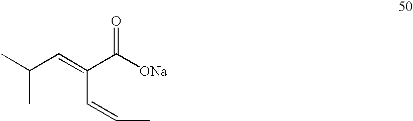

3.62 g of the compound 50 was prepared according to the procedure described above for compound 21 from 3.7 g of compound 49.

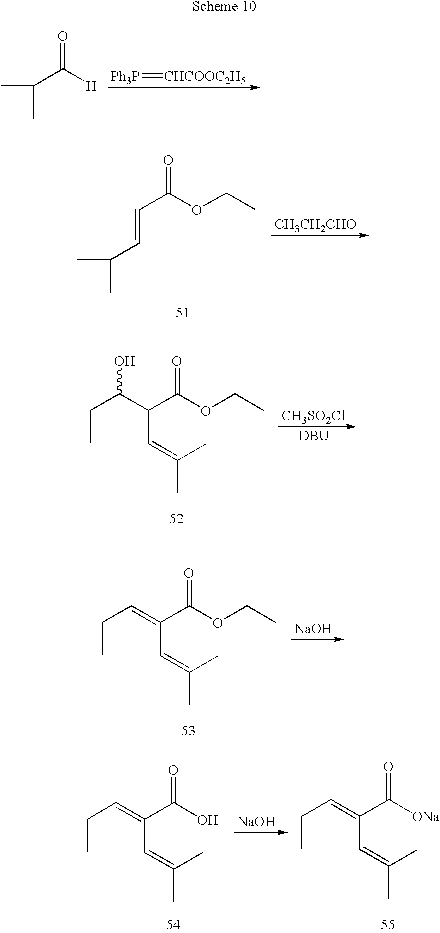

The synthesis of compound 55 is outlined in Scheme 10. The starting ester 51 was prepared from isobutyraldehyde under Wittig reaction's conditions. The ester 51 reacted with propanal under basic conditions to afford alcohol 52. The sodium salt 55 was obtained under the conditions for the preparation of compound 50.

### Preparation of ethyl (2E)-4-methylpent-2-enoate (51)

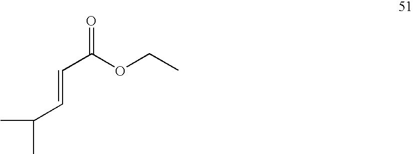

A mixture of (carbethoxymethylene)triphenylphosphorane (52.26 g) in methylene chloride (140 ml) was treated slowly with a solution of 2-methylpropionaldehyde (5.41 g) in methylene chloride (15 ml) and the reaction mixture was stirred at room temperature for 40 hours. The solvent was evaporated under reduced pressure and the residue obtained was washed with hexane and purified by column on silica gel (hexane:ethyl acetate=10:1) to give 10.08 g of compound 51 as a colourless oil.

### Preparation of ethyl 2-(1-hydroxypropyl)-4-methylpent-3-enoate (52)

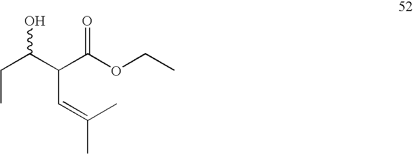

2.04 g (bp 85-89° C./0.6 mmHg) of compound 52 was prepared according to the procedure described above for preparing product 47 from 2.53 g of ethyl (2E)-4-methylpent-2-enoate and a solution of 2-methylbutyraldehyde (1.53 g) in tetrahydrofuran (5 ml) and a standard work-up procedure under acidic conditions.

### Preparation of (2E)-2-(2-methylprop-enyl)pent-2-enoic acid (54)

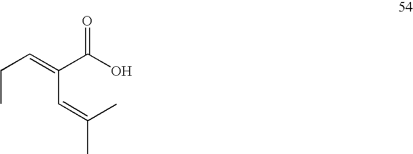

2.03 g of compound 54 was prepared according to the procedure described above for the preparation of product 49 from 3.62 g of compound 52.

¹H NMR (300 MHz, CDCl₃): 11.89 (s, 1H), 6.86 (t, 1H), 5.69 (bs, 1H), 2.08 (m, 2H), 1.81 (s, 3H), 1.51 (s, 3H), 0.99 (t, 3H)

### Preparation of sodium (2E)-2-(2-methylprop-enyl)pent-2-enoate (55)

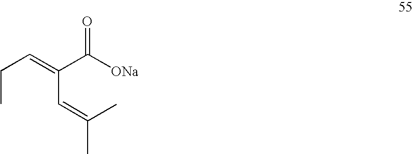

750 mg of compound 55 was prepared according to the procedure described for the preparation of product 50 from 798 mg of product 54.

### EXAMPLE 5

### Pharmacological and Toxicological Testing

Anticonvulsant testing was conducted at the antiepileptic screening facility of the National Institute for Neurological Disorders and Stroke in Rockville, Md. Initial tests were done in mice (i.p.) followed by oral and i.p. administration to rats. Neurotoxicity was evaluated with the rotarod test. Maximal electroshock (MES) and subcutaneous methylene tetrazole (SCMET) or pentylene tetrazole (PTZ) were the most common tests performed. Typical procedures are described below.

MES Assay. Male CD1/CR mice weighing from 25-35 g are administrated test compounds 15 minutes prior to MES. Mice are challenged by pulsed electrical stimulation (50 mA, 0.4 s duration, pulse width 0.5 ms, 60 pulses/sec) via corneal electrodes to induce seizure. Mice are observed post-stimulation for the onset of tonic seizures, and considered to have a tonic seizure only if there is a prolonged extension (>90° from plane of body) of the hind legs. Mice that do not have a seizure, are considered to be protected. Ten mice are used in each group.

SCMET (PTZ85) Assay. Male CD1/CR mice weighing from 25-35 g are administrated test compounds (range of 5 doses) 15 minutes prior to PTZ. PTZ is administrated subcutaneously, just caudal to the cranium, at a dose of 85 mg/kg. Animals are then caged individually and observed for 15 minutes. The occurrence and latency to clonic and/or tonic convulsions are recorded. The mice are used once. Ten mice are used in each dose group. An animal is considered to be unprotected if it shows a 5s clonus with loss of balance. ED₅₀ is determined from a graph of percentage protection vs log(dose) following the known method of Litchfield¹⁸, where percentage protection refers to the percent of animals in each dose group which are protected against seizures.

Rotorod Test. Acute drug induced neurotoxicity is detected in mice using the standard rotorod test. An untreated mouse, when placed on a 6 rpm rotation rod, can maintain its equilibrium for a prolonged period of time. Drug induced neurological impairment is demonstrated by the mouse's inability to maintain equilibrium for one minute in each of three trials.

A number of VPA analogues and their sodium salts were tested for their efficacy as anticonvulsants and for their toxicity using the MES Assay and rotarod test. In particular, the following compounds (as shown in the structures below) were tested: 332059U (sodium salt of VPA), 325071 (known compound, tested for comparison), 325073A (metabolite of VPA), 332060U (4,4-difluoro-2-propylpentanoate, fluorinated analogue of VPA), 341031, and 341032U (conjugated (E,Z)-2,3′-diene VPA analogues). The results of the tests are summarized in Table 2.

TABLE 2
Time to Peak Effect: Selected data obtained on Rats after Oral
administration.

| | |
|---|---|
| 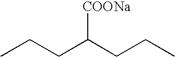 | 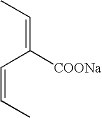 |
| Add ID: 332059U (VPA) | Add ID; 325071, (E,Z)-2,3′-diene |
| Sodium 2-propylpentanoate | Sodium (2E,3Z)-2-ethylidenpent-3-enoate |
| 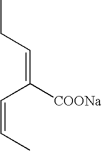 | 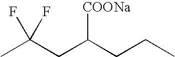 |
| Add ID: 325073A, (E,Z)-2,3′-diene | Add ID: 332060U, 4,4-difluoro-VPA, (7) |
| Sodium (2E)-2-[(1Z)-prop-1-en-1-yl]pent-2-enoate | Sodium 4,4-difluoro-2-propylpentanoate |
| 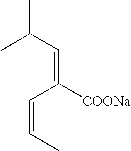 | 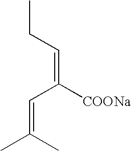 |
| Add ID: 341031, (E,Z)-2,3′-diene, (50) | Add ID: 341032U, (E,Z)-2,3′-diene, (55) |
| Sodium (2E)-4-methyl-2-[(1Z)-prop-1-en-1-yl]pent-2-enoate | Sodium (2E)-2-(2-methylprop-1-en-1-yl)pent-2-enoate |

| | | Dose | # | 0.25 h | 0.5 h | 1.0 h | 2.0 h | 4.0 h | 6.0 h | 8.0 h | 24 h | 36 h |
|---|---|---|---|---|---|---|---|---|---|---|---|---|
| Add ID | Test | (mg/kg) | Dths | N/F | N/F | N/F | N/F | N/F | N/F | N/F | N/F | N/F |
| 332059A | Sodium Valproate | | | | | | | | | | | |
| | MES | 300 | | 0/4 | 1/4 | 1/4 | 0/4 | 0/4 | / | / | / | / |
| | MES | 400 | | / | 1/4 | 2/4 | 0/4 | / | / | / | / | / |
| | TOX | 300 | | 0/4 | 0/4 | 0/4 | 0/4 | 0/4 | / | / | / | / |
| | TOX | 400 | | / | 0/4 | 0/4 | 0/4 | / | / | / | / | / |
| 325071A | Sodium (2E,3Z)-2-ethylidenpent-3-enoate | | | | | | | | | | | |
| | MES | 80 | | 0/4 | 0/4 | 0/4 | 0/4 | 0/4 | / | / | / | / |
| | MES | 200 | | 0/4 | 1/4 | 0/4 | 1/4 | 0/4 | / | / | / | / |
| | SCMET | 80 | | 0/4 | 0/4 | 1/4 | 0/4 | 0/4 | / | / | / | / |
| | SCMET | 200 | | 2/4 | 0/4 | 0/4 | 1/4 | 1/4 | / | / | / | / |
| | TOX | 200 | | 0/8 | 0/8 | 0/8 | 0/8 | 0/4 | / | / | / | / |
| 325073A | Sodium (2E)-2-[(1Z)-prop-1-en-1-yl]pent-2-enoate | | | | | | | | | | | |
| | MES | 50 | | 2/4 | 0/4 | 0/4 | 0/4 | / | / | / | / | / |
| | SCMET | 75 | | 1/4 | 0/4 | 0/4 | 0/4 | 0/4 | / | / | / | / |
| 332060U | Sodium 4,4-difluoro-2-propylpentanoate | | | | | | | | | | | |
| | SCMET | 100 | | 1/4 | 1/4 | 0/4 | 0/4 | 0/4 | / | / | / | / |
| | SCMET | 200 | | 1/4 | 1/4 | / | / | / | / | / | / | / |
| | SCMET | 360 | | 3/4 | 0/4 | 2/4 | 1/4 | 0/4 | | | | |
| | TOX | 100 | | 0/4 | 0/4 | 0/4 | 0/4 | 0/4 | / | / | / | / |
| | TOX | 200 | | 0/4 | 0/4 | / | / | / | / | / | / | / |
| | TOX | 360 | | 0/4 | 0/4 | 0/4 | 0/4 | 0/4 | | | | |
| 341031U | Sodium (2E)-4-methyl-2-[(1Z)-prop-1-en-1-yl]pent-2-enoate | | | | | | | | | | | |
| | MES | 200 | | 3/4 | 2/4 | 0/4 | 0/4 | 0/4 | / | / | / | / |
| | TOX | 200 | | 0/4 | 0/4 | 0/4 | 0/4 | 0/4 | / | / | / | / |
| 341032U | Sodium (2E)-2-(2-methylprop-1-en-1-yl)pent-2-enoate | | | | | | | | | | | |
| | MES | 200 | | 4/4 | 3/4 | 1/4 | 0/4 | 0/4 | / | / | / | / |
| | TOX | 200 | | 3/4 | 4/4 | 1/4 | 0/4 | 0/4 | / | / | / | / |

N/F = number of animals protected relative to number tested.
TOX: N/F = number of animals that failed the rotarod test to numbers tested.
MES = Maximal Electroshock.
SCMET = Subcutaneous Methylene Tetrazole

The data show that the difluoro analogue (332060U, (4,4-difluoro-2-propylpentanoate, fluorinated analogue of VPA)) is effective at doses that do not demonstrate neurotoxicity. The diene analogues 341031U and 341032U are more potent than VPA, but demonstrate that the positioning of groups appears to be important to avoid overt neurotoxicity.

The compounds of interest were tested at an equivalent dose of 1800 micromoles on male cd1/cr mice using the PTZ85 Assay to induce clonic (repetitive seizures). Both the latency (time of seizure onset) and the number of animals protected increased with the size of the ring, or with increased lipophilicity.

TABLE 3
Latency of Clonic Seizures at 1800 micromoles.

| | | | Number of Mice |
|---|---|---|---|
| | | | Protected |
| | | | Against Clonic |
| | | | Seizures |
| Compound | Seconds | SEM | (Out of 10) |
| Control (Saline) | 209 | 40 | 0 |
| 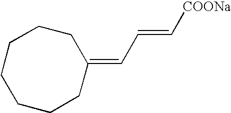 | 855 | 45 | 9 |
| 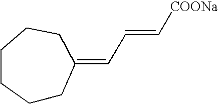 | 778 | 68 | 7 |
| 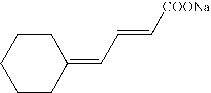 | 661 | 86 | 4 |
| 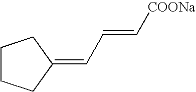 | 589 | 99 | 2 |

SEM = Standard Error of the Mean.

The data indicate the importance of lipophilicity of the molecule to the observed potency, a property consistent with other VPA analogues¹³.

### EXAMPLE 6

### Pharmacological Studies of (E,Z)-2,3′-diene VPA

These compounds were investigated for anticonvulsant activity in mice¹³ and rats (see results in Table 4) and found to be equivalent in potency to VPA.

TABLE 4
Mean effective doses against PTZ-induced seizures and the
slopes of the log dose-response plots for each compound
tested i.p. in male Sprague-Dawley rats (n = 8).

| COMPOUND | ED50, mg/kg | SLOPE |
|---|---|---|
| VPA | 158 (144-187) | 1.2 (1.1-1.3) |
| (E)-2-ENE VPA | 185 (156-199) | 1.2 (1.0-1.3) |
| (E,Z)-2,3′-DIENE VPA | 168 (154-196) | 1.2 (0.8-1.8) |

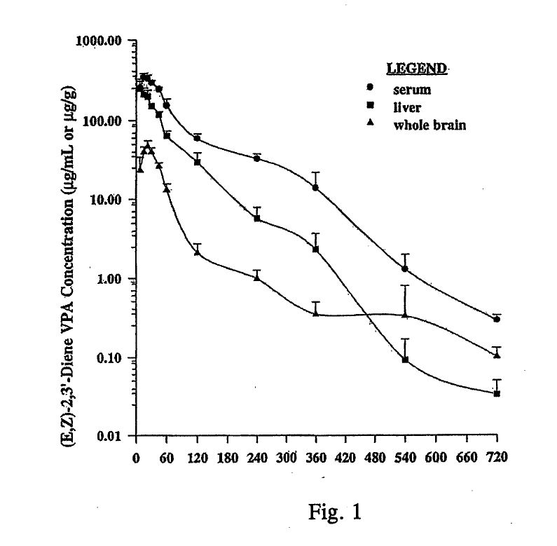

The compound (E,Z)-2,3′-diene VPA has a very favorable anticonvulsant activity profile with respect to VPA (see Tables 2 and 4). Pharmacokinetic studies of (E,Z)-2,3′-diene VPA in the rat demonstrated rapid distribution to the brain, yet a significantly reduced affinity for the liver when compared to VPA (FIG. 1, Table 5). The (E)-2-ene VPA metabolite had similar properties and at one time was being developed as a less hepatotoxic and nonteratogenic alternative to VPA¹⁴. The (E,Z)-2,3′-diene VPA appears to share the same properties. Comparative tissue distribution data for VPA and the unsaturated metabolites in rats are described in Table 5. As can be seen in Table 5, VPA has a great propensity to accumulate in the liver of rats. On the other hand, the unsaturated metabolites, (E)-2-ene VPA and (E,Z)-2,3′-diene-VPA have markedly reduced affinities for liver.

TABLE 5
Area under the curve values (AUC_{0-10 h}) in plasma and liver
following the i.p. administration of VPA, (E)-2-ene VPA and
(E,Z)-2,3′-diene VPA in equivalent doses(sodium salts)
of 150 mg/kg to male Sprague-Dawley rats (n = 8).
AUC_{0-10 h} [ug · h/g or ml (SD)]

| | | | (E,Z)-2,3′- |
|---|---|---|---|
| | VPA | (E)-2-ene VPA | diene VPA |
| PLASMA | 455 (68) | 497 (38) | 518 |
| LIVER | 854 (124) | 384 (63) | 241 |

As will be apparent to those skilled in the art in the light of the foregoing disclosure, many alterations and modifications are possible in the practice of this invention without departing from the scope thereof. Accordingly, the scope of the invention is to be construed in accordance with the substance defined by the following claims.

### LIST OF REFERENCES

1. Dreifuss, F. E., Santili, N., Langer, D. H., Sweeny, K. P., Moline, K. A., Menander, K. B., Valproic acid hepatic fatalities: a retrospective review, Neurology, 37, 379-385 (1987).
2. Kesterson, J. W., Granneman, G. R., Machinist, J. M., The hepatotoxicity of valproic acid and its metabolites in rats. I. Toxicologic, biochemical and histopathologic studies, Hepatology, 4, 1143-1152 (1984).
3. Kingsley, E.; Gray, P.; Tolman, K. G.; Tweedale, R. The toxicity of metabolites of sodium valproate in cultured hepatocytes, J. Clin. Pharmacol., 23, 178-185 (1983).
4. Kassahun, K., Farrell, K., Abbott, F. S., Identification and characterization of the glutathione and N-acetylcysteine conjugates of (E)-2-propyl-2,4-pentadienoic acid, a toxic metabolite of valproic acid, in rats and humans, Drug Metab. Dispos., 19, 525-535 (1991).
5. Genton, P., Gelisse, P., Valproic Acid, Adverse Effects, in Antiepileptic Drugs, 5th edition, Rene H Levy et al. eds., Lippincott Williams and Wilkins, NY, 2002, p 837.
6. Abbott, F. S., Anari, M. R., “Chemistry and biotransformation” in Valproate, Milestones in Drug Therapy, W. Loscher, ed., Birhauser Verlag, Basel, 1999, p. 47.
7. Radatz, M.; Nau, H. “Toxicity” in Valproate, Milestones in Drug Therapy, W. Loscher, ed., Birhauser Verlag, Basel, 1999, p. 115.
8. Tabatabaei, A. R., Thies, R. L., Farrell, K., Abbott, F. S., A rapid in vitro assay for evaluation of metabolism-dependent cytotoxicity of antiepileptic drugs on isolated human lymphocytes, Fundam Appl Toxicol., 37, 181-189 (1997).
9. Winn, L. M., Wells, P. G., Maternal administration of superoxide dismutase and catalase in phenyloin teratogenicity, Free Radic, Biol. Med., 26, 266-274 (1999).
10. Tang, W., Borel, A. G., Fujimiya, T., Abbott, F. S., Fluorinated analogues as mechanistic probes in valproic acid (VPA) hepatotoxicity: Hepatic microvesicular steatosis and glutathione status, Chem. Res. Toxicol., 8, 671-682 (1995).
11. Tang, W., Abbott, F. S., Characterization of thiol conjugated metabolites of 2-propyl-4-pentenoic acid (4-ene-VPA), a toxic metabolite of valproic acid, by ionspray tandem mass spectrometry, J. Mass Spectrom., 31, 926-936 (1996).
12. Tang, W., Palaty, J., Abbott, F. S., Time course of α-fluorinated valproic acid in mouse brain and serum and its effect on synaptosomal GABA levels in comparison to valproic acid, J. Pharmacol. Exp. Therap, 282, 1163-1172 (1997).
13. Palaty, J., Abbott, F. S. Structure-activity relationships of unsaturated analogues of valproic acid, J. Med. Chem., 38, 3398-3406 (1995).
14. Düsing, R. H., Single dose tolerance and pharmacokinetics of 2-n-propyl-(2E)-pentenoate Δ2E-valproate) in healthy male volunteers, Pharm. Weekbl., 14, 152-158 (1992).
15. Elmazar, M. M. A., Hauck, R.-S., Nau, H., Anticonvulsant and neurotoxic activities of twelve analogues of valproic acid, J. Pharm. Sci., 82, 1255-1258 (1993).
16. Hauck, R. S., Nau, H., The enantiomers of the valproic acid analog 2-n-propyl-4-pentynoic acid (4-yn-VPA): asymmetyric synthesis and highly stereoselective teratogenicity in mice, Pharm. Res., 9, 850-855 (1992).
17. Nau, H., Enantioselective synthesis of valproic acid analogs. Application: US 2002-175009. Priority: US 2000-175826; US 2001-760045. CAN 137:325154 AN 2002:814899.
18. Litchfield, J. T., Wilcoxon, F. J. Phar. Exp. Ther., 1949, 96, 99.
19. Tong, V., Chang, T. K. H., Chen, J., Abbott, F. S., The effect of valproic acid on hepatic and plasma levels of 15-F2t-Isoprostane in rats, Free Radic. Biol. Med., 34, 1435-1446 (2003).
20. Sokol, R. J., Straka, M. S., Dahl, R., Devereaux, M. W., Yerushalmi, B., Gumpricht, E., Elkins, N., Everson, G., Role of oxidant stress in the permeability transition induced in rat hepatic mitochondria by hydrophobic bile acids, Pediatric Research, 49, 519-531 (2001).
21. Qu, B., Li, Q-T, Wong, K. P., Tan, T. M. C., Halliwell, B., Mechanism of clofibrate hepatotoxicity: Mitochondrial damage and oxidative stress in hepatocytes, Free Radic. Biol. Med., 31, 659-669 (2001).
22. Tang, W., Abbott, F. S., A comparative investigation of 2-propyl-4-pentenoic acid (4-ene-VPA) and its α-fluorinated analogue in: phase II metabolism and pharmacokinetics, Drug Metab. Dispos., 25, 219-227 (1997).
23. Tsunoda, T., Suzuki, M., Noyori, R. A., A facile procedure for acetalization under aprotic conditions, Tetrahedron Lett., 21, 1357-1358 (1980).
24. Ohshima T., Kagechika, K., Adachi, M., Sodeoka, M., Shobasaki, M., Asymmetric Heck Reaction—Carbanion Capture Process. Catalytic Asymmetric Total Synthesis of (−)-Δ9(12)-Capnellene, J. Am. Chem. Soc., 118, 7108-7117 (1996).
25. Aubert, C., Bergue, J.-P., Charpentier-Morize, M., Nee, G., Langlois, B., Alkylation du trifluoacetylacetate d'ethyle methode generale d'acces aux trifluoromethylcetones. 2eme partie: Alkylations indirects tu TFAAE, J. Fluorine Chem., 44, 377-394 (1989).
26. Montes de Lopez-Cepero, I., Santiago, A., Larson, G. L., A Synthesis of 2,2-ethylenedioxy-5-ketones, Synth. Commun., 16, 705-711 (1986).
27. Kurihara, M., Hakamata, W., Convenient preparation of cyclic acetals, using diols, TMS-source, and a catalytic amount of TMSOTF, J. Org. Chem., 68, 3413-34515 (2003).
28. Middleton, W. J., New fluorinating reagents. Dialkylaminosulfur fluorides, J. Org. Chem., 40, 574-578 (1975).
29. Middleton, W. J., Bingham, E. M., α,α-Difluoroarylacetic acids: Preparation from (diethylamino)sulfur trifluoride and α-oxoarylacetates, J. Org. Chem., 45, 2883-2887 (1980).
30. Lal, G. S., Pez, G. P., Pesaresi, R. J., Prozonic, F. M., Cheng, H., Bis(2-methoxyethyl)aminosulfur trifluoride: A new broad-spectrum deoxofluorinating agent with enhanced thermal stability, J. Org. Chem. 64, 7048-7054 (1999).
31. Lal, G. S., Pez, G. P., Pesaresi, R. J., Prozonic, F. M., Bis(2-methoxyethyl)amino sulfur trifluoride: A new broad-spectrum deoxofluorinating agent with enhanced thermal stability, Chem. Commun., 215-216 (1998).
32. Singh, R. P., Majumder, U., Shreeve, J. M., Nucleophilic Di- and Tetrafluorination of dicarbonyl compounds, J. Org. Chem., 66, 6263-6267 (2001).
33. Tsuji, J., Synthetic applications of the palladium-catalyzed oxidation of olefins to ketones, Synthesis, 369-384 (1983).
34. Tsuji, J., Mizatani, K., Shimzu, I., Yamamoto, K., Synthesis of 2,15-hexadecanedione, a precursor of muscone, from butadiene, Chem. Lett., 773-774 (1976).
35. Tsuji, J., Mizatani, K., Yamamoto, K., Convenient general synthetic method for 1,4- and 1,5-diketones by palladium catalyzed oxidation of α-allyl and α-butenyl ketones, Tetrahedron Lett., 34, 2975-2976 (1976).
36. Brun, E. M., Gil. S., Mestres, R., Parra, M., Regioselective alkylation of lithium dienediolates of α,β-unsaturated carboxylic acids, Synthesis, 1160-1165 (2000).
37. Brun, E. M., Gil. S., Mestres, R., Parra, M., New conditions for the generation of dianions of carboxylic acids, Tethedron Lett., 39, 5443-5446 (1998).
38. Yoshida, Y., Sakakura, Y., Aso, N., Okada, S., Tanabe, Y., Practical and efficient methods for sulfonylation of alcohols using Ts(Ms)Cl/Et3N and catalytic Me3N.HCl as combined base: Promising alternative to traditional pyridine, Tetrahedron, 55, 2183-2192 (1999).
39. Wakabayashi, T., Mori, K., Kobayashi, S., Total synthesis and structural elucidation of Khafrefungin, J. Am. Chem. Soc., 123, 1372-1375 (2001).
40. Lee, R. D., Kassahun, K., Abbott, F. S., Stereoselective synthesis of the diunsaturated metabolites of valproic acid, J. Pharm. Sci., 78, 667-671 (1989).
41. Maryanoff, B. E.; Reitz, A. B. Chem. Rev. 1989, 89, 863-927 and references therein.
42. (a) DiBiase, S. A.; Lipisko, B. A.; Haag, A.; Wolak, R. A.; Gokel, G. W. J. Org. Chem. 1979, 44, 4640-4649; (b) Wu, K. M.; Midland, M. M.; Okamura, W. H. J. Org. Chem. 1990, 55, 4381-4392; (c) Clive, D. L. J.; Farina, V.; Beaulieu, P. L. J. Org. Chem. 1982, 47, 2572-2582; (d) Ono, N.; Miyake, H.; Tanikaga, R.; Kaji, A. J. Org. Chem. 1982, 47, 5017-5019.
43. Takacs, J. M.; Jaber, M. R.; Clement, F.; Walters, C. J. Org. Chem. 1998, 63, 6757-6760.
44. Bonadies, F.; Cardilli, A.; Lattanzi, A.; Orelli, L. R.; Scettri, A. Tetrahedron Letters 1994, 35, 3383-3386.
45. Bennani, Y. L.; Boehm, M. F. J. Org. Chem. 1995, 60, 1195-1200.
46. Motoyoshiya, J. Trends in Organic Chemistry, 1998, 7, 63-73 and references therein.
47. Pihko, P. M.; Salo, T. M. Tetrahedron Letters, 2003, 44, 4361-4364 and references therein.
48. Parisi, M. F., Gattuso, G., Notti, A., and Raymo, F. M., J. Org. Chem., 1995, 60, 5174-5179.

1. A compound selected from the group consisting of compounds represented by the formula (I) and stereoisomers and pharmaceutically acceptable salts thereof 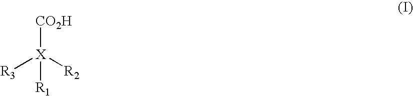
wherein said compound is an analogue of valproic acid and comprises between 5 and 13 carbon atoms;
wherein X═C;
wherein R₁ is optionally present and when present is either H or F;
wherein, when R₁ is present, R₂ and R₃ are selected from the group consisting of a linear or branched C1 to C6 alkyl, a linear or branched C2 to C6 n-ene hydrocarbyl (where n=1-5), a linear or branched C1 to C6 n-yne hydrocarbyl (where n=1-5), a linear or branched C1 to C5 ether, a linear or branched C1 to C6 ketone, and —CHₓ-A where A=cyclic C3 to C8 hydrocarbyl and x=0-3;
wherein, when R₁ is H, at least one of R₂ and R₃ are selectively fluorinated;
wherein, when R₁ is F, R₂ and R₃ comprise linear or branched alkenyl groups;
wherein, when R₁ is not present, R₂ is H, there is a double bond between R₃ and X, and R₃ is 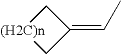
wherein n is 1 to 10;
or when R₁ is not present, there is a single bond between X and R₂, R₂ is 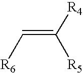
wherein R₄, R₅ and R₆ are selected from the group consisting of H, methyl, ethyl, F, NH₂, cyclopropyl, CF₃, and saturated or unsaturated cyclic (C3 to C8) hydrocarbyl, there is a double bond between R₃ and X, and R₃ is 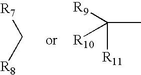
wherein R₇ and R₈ are selected from the group consisting of H, methyl, ethyl, F, NH₂, cyclopropyl and CF₃, and R₉, R₁₀, and R₁₁ are selected from the group consisting of H, methyl, ethyl, F, NH₂, cyclopropyl and CF₃.

2. The compound as defined in claim 1, wherein the total number of carbon atoms in said compound is between 6 and 10.

3. The compound as defined in claim 2, wherein the total number of carbons in said compound is 8.

4. The compound as defined in claim 1, wherein said compound has multiple sites of alkene or alkyne unsaturation.

5. The compound as defined in claim 1, wherein R₂ and R₃ are selected from the group consisting of propyl, propenyl and propynyl substituents.

6. The compound as defined in claim 5, wherein R₂ and R₃ are selectively fluorinated at one or more secondary carbon atoms.

7. The compound as defined in claim 6, wherein at least one or more of said secondary carbon atoms is monofluorinated.

8. The compound as defined in claim 6, wherein at least one or more of said secondary carbon atoms is difluorinated.

9. The compound as defined in claim 1, wherein R₂ and R₃ are selectively fluorinated linear or branched alkyl or alkenyl groups having 1 to 6 carbons atoms.

10. The compound as defined in claim 1, wherein R₁ is H and R₂ and R₃ each comprise an optionally substituted alkyl group, said compound having the formula (II) 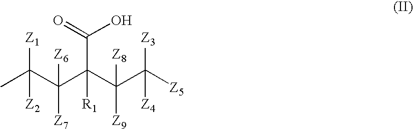
wherein at least one of Z₁, Z₂, Z₃, Z₄, Z₅, Z₆, Z₇, Z₈, and Z₉ is F and Z₅ is CH₃.

11. The compound as defined in claim 10, wherein Z₁ and Z₂ are F and Z₃ and Z₄ are H.

12. The compound as defined in claim 10, wherein Z₁, Z₂, Z₃ and Z₄ are F.

13. The compound as defined in claim 10, wherein Z₁, Z₂, Z₈, and Z₉ are F.

14. The compound as defined in claim 10, wherein Z₁ and Z₂ are F and Z₃ and Z₄ together form a ═O group.

15. The compound as defined in claim 10, wherein Z₆ and Z₇ are F and Z₈ and Z₉ are H.

16. The compound as defined in claim 10, wherein Z₆, Z₇, Z₈ and Z₉ are F.

17. The compound as defined in claim 10, wherein Z₆ and Z₇ are F and Z₈ and Z₉ together form a ═O group.

18. The compound as defined in claim 1 wherein R₁ is F and R₂ and R₃ each comprise an optionally substituted alkenyl group, said compound having the formula (III)

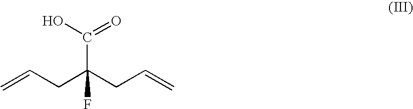

19. The compound as defined in claim 5 wherein the terminal carbon of the propyl, propenyl and propynyl substituent is fluorinated.

20. The compound as defined in claim 1 wherein one of R₂ and R₃ comprises a 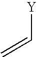
moiety, wherein Y is selected from the group consisting of CF₃, CF₂H, and CFH₂, and the other of R2 and R3 comprises a linear or branched alkyl group.

21. The compound as defined in claim 1, wherein said compound comprises an optionally fluorinated dialkenyl chain.

22. The compound as defined in claim 1, wherein said compound comprises a C1 to C3 hydrocarbyl group.

23. The compound as defined in claim 1, wherein n is between 4 and 8.

24. The compound as defined in claim 23, wherein n is 4 or 5.

25. The compound as defined in claim 1, wherein said compound is a diene having an E,Z configuration.

26. The compound as defined in claim 1, wherein R₁ is absent and R₂ and R₃ are unsaturated groups, said compound containing a backbone of formula IV 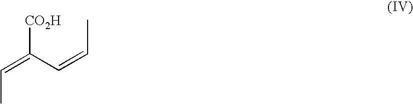
wherein the backbone is optionally substituted by H, F, Me, Et, NH₂, or C1 to C3 hydrocarbyl groups.

27. The compound as defined in claim 1, wherein said compound is selected from the group consisting of
4,4-difluoro-2-propylpentanoic acid,
3,3-difluoro-2-propylpentanoic acid,
2,3,3-trifluoro-2-propylpentanoic acid,
2,4,4-trifluoro-2-propylpentanoic acid,
2-(3,3,3-trifluoropropyl)-4,4-difluoropentanoic acid,
2-(3,3,3-trifluoropropyl)-3,3-difluoropentanoic acid,
2-(2,2-difluoropropyl)-4,4-difluoropentanoic acid,
2-(1,1-difluoropropyl)-3,3-difluoropentanoic acid,
2-(2,2-difluoropropyl)-3,3-difluoropentanoic acid,
4,4-difluoro-2-(2-oxopropyl)pentanoic acid,
4,4-difluoro-2-(2-oxapropyl)pentanoic acid,
2-(2,2-difluoropropyl)pent-3-ynoic acid,
2-(1,1-difluoropropyl)pent-3-ynoic acid,
(3E)-2-(2,2-difluoropropyl)pent-3-enoic acid,
(3Z)-2-(2,2-difluoropropyl)pent-3-enoic acid,
2-(2,2-difluoropropyl)pent-4-enoic acid,
2-(2,2-difluoropropyl)pent-4-enoic acid,
(3E)-2-(1,1-difluoropropyl)pent-3-enoic acid,
(3Z)-2-(1,1-difluoropropyl)pent-3-enoic acid,
2-(1,1-difluoropropyl)pent-4-enoic acid,
2-(1,1-difluoropropyl)pent-4-enoic acid,
2-Allyl-2-fluoropent-4-enoic acid,
(2E)-4,4-difluoro-2-propylpent-2-enoic acid
(2Z)-4,4-difluoro-2-propylpent-2-enoic acid,
2-propyl-3-(trifluoromethyl)but-3-enoic acid,
2-iso-propyl-3-(trifluoromethyl)but-3-enoic acid,
2-butyl-3-(trifluoromethyl)but-3-enoic acid,
2-sec-butyl-3-(trifluoromethyl)but-3-enoic acid,
4,4-difluoro-(2-cyclopropylmethyl)pentanoic acid,
4,4-difluoro-(2-cyclobutylmethyl)pentanoic acid,
4,4-difluoro-(2-cyclopentylmethyl)pentanoic acid,
4,4-difluoro-(2-cyclohexylmethyl)pentanoic acid,
3,3-difluoro-(2-cyclopropylmethyl)pentanoic acid,
3,3-difluoro-(2-cyclobutylmethyl)pentanoic acid,
3,3-difluoro-(2-cyclopentylmethyl)pentanoic acid,
3,3-difluoro-(2-cyclohexylmethyl)pentanoic acid,
(2E)-4-Cyclopentylidenebut-2-enoic acid,
(2E)-4-Cyclohexylidenebut-2-enoic acid,
(2E)-4-Cycloheptylidenebut-2-enoic acid,
(2E)-4-Cyclooctylidenebut-2-enoic acid,
(2Z)-4-cyclopentylidenebut-2-enoic acid,
(2Z)-4-Cyclohexylidenebut-2-enoic acid,
(2Z)-4-Cycloheptylidenebut-2-enoic acid,
(2Z)-4-Cyclooctylidenebut-2-enoic acid,
(2E)-4-methyl-2-[(1 Z)-prop-1-enyl])pent-2-enoic acid,
(2E)-2-(2-methylprop-1-enyl)pent-2-enoic acid,
(2E)-2-(2-methylprop-1-en-1-yl)pent-2-enoic acid, and
(2E)-4-methyl-2-[(1 Z)-prop-1-en-1-yl]pent-2-enoic acid.

28. A method of treating a patient having a condition responsive to valproic acid therapy comprising administering a therapeutically effective amount of a compound according to claim 1.

29. The method of claim 28, wherein said condition is a neuroaffective disorder selected from the group consisting of seizures, epilepsy, bipolar disease and migraine headaches.

30. A method of reducing seizure activity in a mammal comprising administering to said mammal a therapeutically effective amount of a compound according to claim 1.

31. A pharmaceutical composition comprising an effective amount of a compound according to claim 1 together with a pharmaceutically effective carrier or at least one pharmaceutically acceptable additive.

32. (canceled)

33. (canceled)

34. (canceled)

35. A prodrug transformable in vivo to a compound according to claim 1.

36. A prodrug according to claim 35, comprising esters or amides of said compound.

37. A prodrug according to claim 35, comprising a salt of said compound.

38. A prodrug according to claim 37, comprising a sodium salt of said compound.

39. A method of treating a patient having a condition responsive to valproic acid therapy comprising administering a prodrug according to claim 35.

40. A method according to claim 39, wherein said condition is a neuroaffective disorder selected from the group consisting of seizures, epilepsy, bipolar disease and migraine headaches.

41. A method of synthesizing an analogue of valproic acid comprising the steps set forth in any one of Schemes 4, 5, 6, 7, 8, 9, and 10.

42. A method of treating a patient having a condition responsive to valproic acid therapy comprising administering a therapeutically effective amount of a compound according to claim 27.

43. A pharmaceutical composition comprising an effective amount of a compound according to claim 27 together with a pharmaceutically effective carrier or at least one pharmaceutically acceptable additive.

44. A prodrug transformable in vivo to a compound according to claim 27.
